(19) United States
(12) Patent Application Publication
(10) Pub. No.: US 2014/0114188 A1
(43) Pub. Date: Apr. 24, 2014

(54) ACOUSTO-OPTIC VIBROMETER

(71) Applicant: Panasonic Corporation, Osaka (JP)

(72) Inventors: Ushio SANGAWA, Nara (JP); Takuya IWAMOTO, Osaka (JP); Yuriko KANEKO, Nara (JP); Takahiro KAMAI, Kyoto (JP); Masahiko HASHIMOTO, Osaka (JP)

(73) Assignee: Panasonic Corporation, Osaka (JP)

(21) Appl. No.: 14/144,784

(22) Filed: Dec. 31, 2013

Related U.S. Application Data
(63) Continuation of application No. PCT/JP2013/003075, filed on May 14, 2013.

(30) Foreign Application Priority Data
May 15, 2012 (JP) 2012-111606

(51) Int. Cl. A61B 5/00

(52) U.S. Cl. CPC A61B 5/0097
USPC 600/437

(57) ABSTRACT

An acousto-optic vibrometer includes: an acoustic wave source to irradiating an object with an acoustic wave; an acoustic lens system which places a scattered wave from the object into a predetermined converged state; an acousto-optic medium portion in which the scattered wave transmits; a sensing light source to emit a sensing light beam in which monochromatic rays of light with different traveling directions are superposed and which is incident on the acousto-optic medium portion; a reference light source to emit a reference light beam in which monochromatic rays of light with different traveling directions are superposed and which is to be superposed on sensing light beam based diffracted light produced by the acousto-optic medium portion; an imaging lens system which converges the diffracted light on which the reference light beam is superposed; and an image receiving section which senses the light converged by the imaging lens system.

This is a continuation of International Application No. PCT/JP2013/003075, with an international filing date of May 14, 2013, which claims priority of Japanese Patent Application No. 2012-111606, filed on May 15, 2012, the contents of which are hereby incorporated by reference.

### BACKGROUND

1. Technical Field

The present application relates to an acousto-optic vibrometer which measures the motion of an object by light and acoustic waves.

2. Description of the Related Art

Recently, the number of patients who suffer from some cardiovascular disease such as heart infarction or brain infarction is expected to rise year after year, and there are increasing demands for techniques for diagnosing those diseases.

One of various pieces of information that can be used effectively to diagnose such a cardiovascular disease is the elastic property of a tissue or organ to be observed via a quick and dynamic behavior of the organ. For example, some people are trying to inspect the elastic property of a diseased region of a person's under test and determine the degree of advancement of arterial sclerosis or the size of the diseased region by checking out the distribution of displacement on the cardiac or arterial wall inside his or her body in a frequency range which is higher than his or her heart rate. Since such an inspection can get done easily and non-invasively, some people propose that an ultrasonic diagnostic apparatus be used after the elastic property has been measured in this manner.

A conventional ultrasonic diagnostic apparatus irradiates an internal body tissue such as an organ with an ultrasonic wave that has been generated externally and detects the ultrasonic wave that has been reflected from the internal body tissue, thereby obtaining a two- or three-dimensional image representing the internal body tissue. Such a conventional ultrasonic diagnostic apparatus is disclosed in Japanese Laid-Open Patent Publication No. 54-34580 (hereinafter, referred to as “Patent Document No. 1”), for example. To transmit and receive ultrasonic waves, the conventional ultrasonic diagnostic apparatus includes a probe with a plurality of ultrasonic transducers. For example, as shown in FIG. 30, the probe may include transducers T₁ through T₁₅ which are arranged one-dimensionally.

In receiving the ultrasonic waves, these transducers T₁ through T₁₅ each receive an ultrasonic wave that has been reflected from the internal body tissue and output an electrical signal. Those received signals are delay-synthesized by a signal processor (not shown in FIG. 30) to generate a single received signal. Supposing the received signal output from a transducer Ti (where i=1, 2, . . . or 15) is identified by Si(t) (where i=1, 2, . . . or 15), the delayed synthesis refers herein to the computation given by A1×S1(t+t1)+A2×S2(t+t2)+, . . . +A15×S15(t+t15), where t indicates the time, ti (where i=1, 2, . . . or 15) indicates the time lag (i.e., time delay), and Ai (where i=1, 2, . . . or 15) indicates the weight (real number). As can be seen, the “delayed synthesis” refers herein to a signal synthesizing method in which the electrical signals output from the respective transducers are added together with weights added to them at respective shifted timings.

As shown in FIG. 30, suppose an ultrasonic wave that has been transmitted from the probe is reflected from a point a₂ to generate a pulsed spherical wave, which sequentially propagates toward the transducers T₁ through T₁₅. In this example, a point in time when the spherical wave reaches the transducer T₅ (which is closer to the point a₂ than any other transducer) is supposed to be a reference time and each of the other transducers Tᵢ is supposed to output an electrical signal at a time delay of τi (where τi>0). If the delayed synthesis described above is carried out on the supposition that ti=τi (where i=1, 2, . . . or 15), then every delay signal Si(t+ti) based on the electrical signal of each transducer becomes a time signal in which a pulsed waveform appears at the same point in time. As a result, the delay-synthesized signal becomes a received signal (time signal) with a tall pulsed waveform.

Suppose a pulsed spherical wave has been created at a point other than a₂, e.g., at a point a₁. In that case, in the delay signal Si (t+ti) of the received signal output from each transducer, the pulsed waveform corresponding to that spherical wave does not appear at the same point in time. The reason is that the distance from a₂ to each transducer is different from the distance from a₁ to that transducer and the two spherical waves will reach each transducer at different points in time. Consequently, in the delay-synthesized received signal, a waveform representing the spherical wave that has come from the point a₁ does not shows any tall pulse signal corresponding to the point a₁.

By generating the received signals in this manner, the time delay ti (where i=1, 2, . . . or 15) is set so as to be sensitive to only a spherical wave that has come from any arbitrary point on the paper on which FIG. 30 is drawn, a pulse signal is transmitted and received from/at the transducers T₁ through T₁₅ at each time delay that has been set, and delay synthesis is carried out at the time delay set on the received signals. In this manner, spherical waves that have come from respective points in the internal body tissue can be detected.

The spherical wave reflected from each of those points has amplitude corresponding to the intensity of reflection, which depends on a difference in the elastic property of the tissue between respective points or a difference in acoustic impedance between the tissues, for example. That is why by analyzing the intensity distribution of the spherical wave in the received signal, a tomographic image representing the internal body tissue can be obtained.

### SUMMARY

The conventional ultrasonic diagnostic apparatus can capture a tomographic image of an internal body tissue or organ from the surface of the subject's body. However, to capture a single ultrasonic image, the delay synthesis signal processing should be carried out roughly as often as the total number of pixels in the image capturing region. That is why to capture a tomographic image at high speeds, a sufficiently high-speed signal processor with a large scale array of analog/digital converters and an arithmetic-logic unit is needed. A retailed high-performance ultrasonic diagnostic apparatus includes a high-speed signal processor of such a large scale, and therefore, can capture tomographic images at a rate of several ten frames per second. Still, it is very difficult for such an apparatus to monitor the vibration state of the heart at as high a frequency as approximately several ten to one hundred Hz as is required in checking its function or to achieve a resolution that is high enough to capture as small a vascular deformation as a few ten μm due to the heartbeat.

Also, even if the probe disclosed in Patent Document No. 1 is used in combination with the signal processing method for detecting a zero-cross point of a received signal as disclosed in Japanese Laid-Open Patent Publication No. 2000-229078 (hereinafter, referred to as “Patent Document No. 2”), a pulse Doppler method or any other phase detection method to be applied to a radar, or any of various calibration methods dedicated to the tissue under test, the vibration state of the blood vessel can be observed as a pulse wave at a frequency of a few hundred Hz. Nevertheless, there is an increasing demand for a general-purpose device that can be used on the spot efficiently enough for clinical purposes. For example, the testing environment still needs to be optimized according to the specific tissue under test.

A non-limiting exemplary embodiment of the present application provides an acousto-optic vibrometer that can shoot the object at high speeds.

To overcome the problem described above, an acousto-optic vibrometer according to an aspect of the present invention includes: an acoustic wave source; an acoustic lens system which places a scattered wave, created by irradiating an object with an acoustic wave that has been emitted from the acoustic wave source, into a predetermined converged state; an acousto-optic medium portion which is arranged so that the scattered wave that has been transmitted through the acoustic lens system is incident on the acousto-optic medium portion; a sensing light source to emit a sensing light beam in which a plurality of monochromatic rays of light with mutually different traveling directions are superposed one upon the other and which is incident on the acousto-optic medium portion at an angle with respect to, and neither perpendicularly nor parallel to, the acoustic axis of the acoustic lens system; a reference light source to emit a reference light beam in which a plurality of monochromatic rays of light with mutually different traveling directions are superposed one upon the other and which is to be superposed on sensing light beam based diffracted light that has been produced by the acousto-optic medium portion; an imaging lens system which converges the diffracted light on which the reference light beam is superposed; and an image receiving section which senses the light that has been converged by the imaging lens system to output an electrical signal.

An acousto-optic vibrometer according to the present disclosure can not only capture high-definition images of the object at high speeds but also measure the object's displacement velocity distribution.

These general and specific aspects may be implemented using a system, a method, and a computer program, and any combination of systems, methods, and computer programs.

Additional benefits and advantages of the disclosed embodiments will be apparent from the specification and Figures. The benefits and/or advantages may be individually provided by the various embodiments and features of the specification and drawings disclosure, and need not all be provided in order to obtain one or more of the same.

### BRIEF DESCRIPTION OF THE DRAWINGS

FIG. 1 illustrates a general configuration for a first embodiment of an acousto-optic vibrometer according to the present invention.

FIG. 2 is a ray diagram showing the function of an acoustic lens system 6 according to the first embodiment.

FIG. 3 illustrates a configuration for a sensing light source 19 according to the first embodiment.

FIG. 4A illustrates a configuration for a uniform illumination optical system 31 according to the first embodiment.

FIG. 4B illustrates another configuration and other rays of light.

FIG. 5 illustrates where a uniformly illuminated plane 43 may be set according to the first embodiment.

FIG. 6 illustrates a configuration and rays of light for a reference light source 23 according to the first embodiment.

FIG. 7 illustrates an exemplary configuration for an acousto-optic modulator 214 according to the first embodiment.

FIG. 8A illustrates how a sensing light beam 14 is diffracted by a plane acoustic wave 9 in the acousto-optic vibrometer of the first embodiment.

FIG. 8B shows a Bragg diffraction condition on a one-dimensional diffraction grating.

FIG. 8C shows how the acoustic pressure distribution on the plane acoustic wave is transferred by Bragg diffraction onto a light amplitude distribution on the wavefront of a diffracted light beam.

FIG. 9A illustrates how the diffracted light 201 is distorted in the y direction in the acousto-optic vibrometer of the first embodiment.

FIG. 9B illustrates the structure of an anamorphic prism for use as an image distortion compensating section 15 in the acousto-optic vibrometer of the first embodiment.

FIG. 10 illustrates how a wedge prism which forms part of an anamorphic prism works.

FIG. 11 illustrates how the acousto-optic vibrometer of the first embodiment needs a light beam in which a large number of plane-wave light beams with mutually different angles of incidence are superposed one upon the other.

FIG. 12A illustrates how a double diffraction optical system works in the field of optics.

FIG. 12B illustrates the acousto-optic system in the acousto-optic vibrometer of the first embodiment.

FIG. 13 illustrates a Doppler shift 233 caused by an object 4 which is displaced with time.

FIG. 14 illustrates how a displacement velocity vector distribution on the object 4 is mirrored in the frequency modulation of a plane wave ultrasonic wave created by the acoustic lens system 6.

FIG. 15 illustrates how the frequency of a +first-order Bragg diffracted light beam created by a plane acoustic wave is increased by the frequency of the plane acoustic wave.

FIG. 16 illustrates how a displacement velocity vector distribution on the object 4 is mirrored in the frequency modulation of luminous points on the real image 18.

FIG. 17 illustrates how the luminous points on the real image 18 turn into a beat light beam by superposing a reference light beam 24.

FIG. 18 illustrates how to measure the displacement velocity vector distribution on the object 4 according to the first embodiment.

FIG. 19 illustrates how to measure the displacement velocity vector distribution on the object 4 as a vector quantity.

FIG. 20A illustrates an exemplary procedure of capturing an image using an acoustic wave and measuring a displacement velocity vector distribution according to the first embodiment.

FIG. 20B illustrates another exemplary procedure of doing that.

FIG. 21 illustrates an exemplary specific configuration for an acousto-optic vibrometer according to the first embodiment.

FIG. 22 illustrates an example in which an acousto-optic vibrometer according to the first embodiment is implemented as an ultrasonic diagnostic apparatus.

FIGS. 23A and 23B show two different incoming directions of a sensing light beam 14 in the acousto-optic vibrometer according to the first embodiment.

FIG. 24 illustrates the structure of a cylindrical lens and a ray of light.

FIG. 25 illustrates an optical system which is implemented as a combination of two cylindrical lenses and which functions as both an image distortion compensating section 15 and as an imaging lens system 16 in the acousto-optic vibrometer of the first embodiment.

FIG. 26 illustrates a configuration for an image distortion compensating section 15 in an acousto-optic vibrometer according to a second embodiment.

FIG. 27 illustrates a configuration for an image distortion compensating section 15 in an acousto-optic vibrometer according to a third embodiment.

FIG. 28 illustrates a configuration for an acousto-optic vibrometer according to a fourth embodiment.

FIG. 29 illustrates a configuration for an acousto-optic vibrometer according to a fifth embodiment.

FIG. 30 illustrates a method of detecting an ultrasonic wave with a probe for use in a conventional ultrasonic diagnostic apparatus.

### DETAILED DESCRIPTION

An aspect of the present invention can be outlined as follows.

An acousto-optic vibrometer according to an aspect of the present invention includes: an acoustic wave source; an acoustic lens system which places a scattered wave, created by irradiating an object with an acoustic wave that has been emitted from the acoustic wave source, into a predetermined converged state; an acousto-optic medium portion which is arranged so that the scattered wave that has been transmitted through the acoustic lens system is incident on the acousto-optic medium portion; a sensing light source to emit a sensing light beam in which a plurality of monochromatic rays of light with mutually different traveling directions are superposed one upon the other and which is incident on the acousto-optic medium portion at an angle with respect to, and neither perpendicularly nor parallel to, the acoustic axis of the acoustic lens system; a reference light source to emit a reference light beam in which a plurality of monochromatic rays of light with mutually different traveling directions are superposed one upon the other and which is to be superposed on sensing light beam based diffracted light that has been produced by the acousto-optic medium portion; an imaging lens system which converges the diffracted light on which the reference light beam is superposed; and an image receiving section which senses the light that has been converged by the imaging lens system to output an electrical signal.

The sensing light beam and the reference light beam may have mutually different frequencies.

The reference light source may include at least one acousto-optic modulator.

The reference light source may include a light scattering plate.

The reference light source may include a fly-eye lens.

The acousto-optic vibrometer may include two optical systems, each including the imaging lens system and the image receiving section.

The reference light source may include a polarizer.

The image receiving section may be a two-dimensional image sensor with a plurality of pixels that are arranged two-dimensionally.

The acousto-optic vibrometer may further include an image processing section which detects, based on the electrical signal, a variation with time in the quantity of light detected by each said pixel of the image receiving section.

The reference light source may include a shutter which controls the time of emittance of the reference light beam.

The acoustic wave source may include at least three acoustic wave sources.

The acousto-optic vibrometer may further include an image distortion correcting section which corrects the distortion of the diffracted light and/or the distortion of the object image represented by the electrical signal.

The image distortion correcting section may include an optical member which increases the cross-sectional area of the diffracted light.

The image distortion correcting section may include an optical member which decreases the cross-sectional area of the diffracted light.

The optical member may include an anamorphic prism.

At least one of the imaging lens system and the optical member may include at least one cylindrical lens.

The image distortion correcting section may correct the distortion of the object image, which is represented by the electrical signal, based on the electrical signal.

The monochromatic rays of light may have a spectrum width of less than 10 nm and may be plane waves, of which the wavefront accuracy is ten times or less of the wavelength at the center frequency of the monochromatic rays of light.

The sensing light source may include at least one fly-eye lens.

The acoustic lens system may include at least one of a refracting acoustic lens and a reflecting acoustic lens.

The acoustic lens system may include at least one acoustic element which is made of a material selected from the group consisting of a nanoporous silica, a fluorine-based inactive liquid, and polystyrene.

The acoustic lens system may include at least one of a focal length adjusting mechanism and a focus position adjusting mechanism.

The imaging lens system may include at least one of a focal length adjusting mechanism and a focus position adjusting mechanism.

The acousto-optic medium portion may include at least one of a nanoporous silica, a fluorine-based inactive liquid, and water.

The optical axis of the sensing light beam emitted from the sensing light source may be adjustable with respect to the acoustic axis of the acoustic lens.

Hereinafter, embodiments of the present invention will be described with reference to the accompanying drawings.

### Embodiment 1

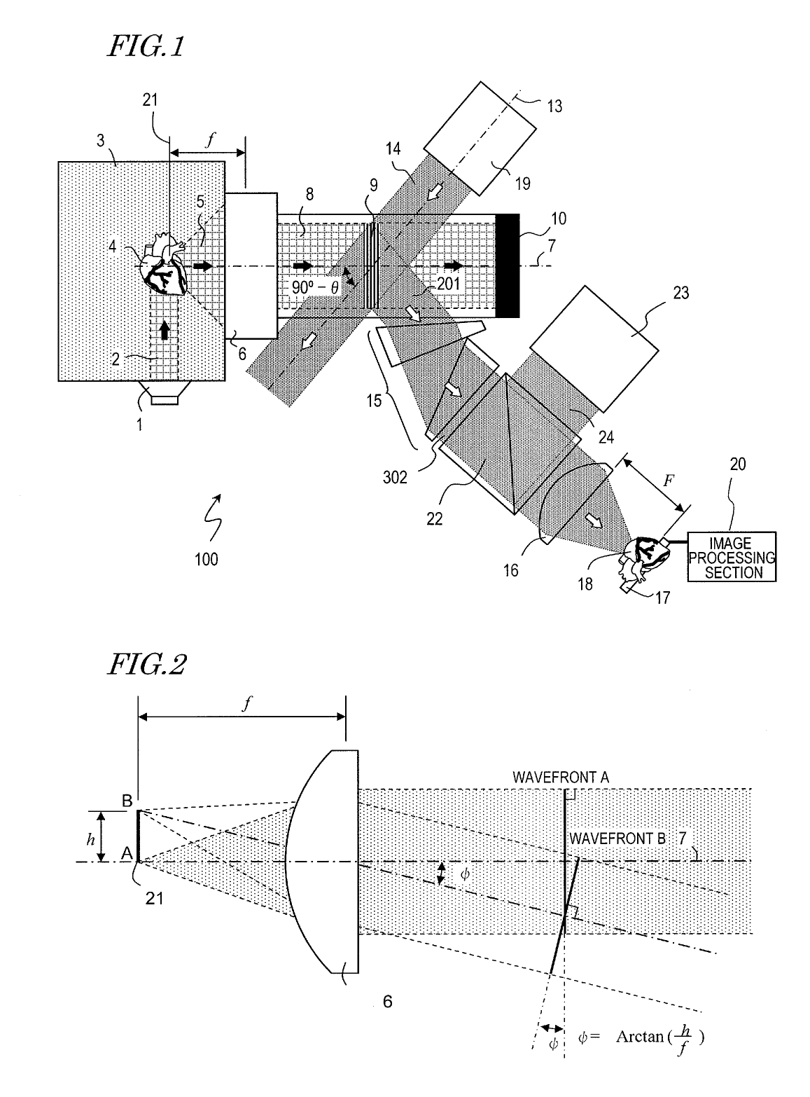

FIG. 1 schematically illustrates a configuration for an acousto-optic vibrometer 100 as a first embodiment. The acousto-optic vibrometer 100 includes an acoustic wave source 1, an acousto-optic medium portion 8, an acoustic lens system 6, a sensing light source 19, a reference light source 23, an imaging lens system 16, and an image receiving section (image forming section) 17. The vibrometer 100 may further include an acoustic wave absorbing end 10, an image distortion compensating section 15 and a beam splitter 22.

An object 4 is arranged in a medium 3 which can propagate an acoustic wave. Examples of such a “medium 3 which can propagate an acoustic wave” include the air and water. A body tissue is also a typical example of such a “medium 3 which can propagate an acoustic wave”. In addition, an elastic body such as a metal or concrete also propagates an acoustic wave, and therefore, can also be used as the medium 3. The object 4 is the object of sensing which has a different modulus of elasticity from the medium 3. Specifically, if the acousto-optic vibrometer 100 is used to observe an internal body tissue of a person under test, the medium 3 is the body tissue and the object 4 is an organ or a tissue which is the object of observation. On the other hand, if this acousto-optic vibrometer 100 is used to carry out a non-destructive test on a physical structure, the medium 3 is a metal or concrete, and the object 4 is a structural defect such as a crack or a hole.

The acoustic wave source 1 and the acoustic lens system 6 are arranged so as to contact directly with the medium 3 or contact indirectly with the medium 3 with some intermediate layer interposed between them. For example, in a situation where an internal body tissue of a person under test is going to be observed, if the acoustic wave source 1 and the acoustic lens system 6 cannot make good contact with the medium 3 due to the surface shape of the medium 3, then the acoustic wave source 1 and the acoustic lens system 6 may contact with the medium 3 via a gel material having a similar acoustic property to that of the medium 3.

The acousto-optic vibrometer 100 irradiates the object 4 with an acoustic wave that has been emitted from the acoustic wave source 1, thereby capturing an optical image of the object 4 as a real image 18. The real image 18 is an image generated by getting the acoustic wave scattered from the object 4. That is to say, to view the real image 18 is equivalent to observing a three-dimensional distribution of moduli of elasticity on the surface and inside of the object 4 from the direction of the acoustic axis 7. More specifically, the real image 18 is an image which is in focus most sharply on the two-dimensional distribution of moduli of elasticity of the object 4 on a plane which intersects at right angles with the acoustic axis 7 and which is located away from the acoustic lens system 6 by the focal length f of the acoustic lens system 6 and which gets more and more blurred (i.e., out of focus), the more distant from the plane. In this respect, the real image 18 is similar to a microscopic image. Such a two-dimensional distribution of moduli of elasticity of the object 4 on a plane which intersects at right angles with the acoustic axis 7, which is located away from the acoustic lens system 6 by the focal length f of the acoustic lens system 6 and where a most detailed image can be obtained is similar to the real image 18.

The real image 18 is an image, in which the light intensity varies at a frequency corresponding to the distribution of displacement velocities on the object 4. The acousto-optic vibrometer 100 gets the distribution of flickering periods of the light intensity of the real image measured by the image receiving section 17, thereby obtaining an image of the object 4 based on the acoustic wave and measuring the distribution of displacement velocities.

1. Configuration of Acousto-Optic Vibrometer 100

(1) Acoustic Wave Source 1

The acoustic wave source 1 radiates an acoustic wave 2 toward the object 4. The frequency of the acoustic wave 2 which is best suited to observe the object 4 is selected according to the elastic property of the object 4 and the environment such as the medium 3 surrounding the object 4. Specifically, if the object 4 is an organ of a person under test, the acoustic wave 2 may be an ultrasonic wave which is used in a known ultrasonic diagnostic apparatus, e.g., an ultrasonic wave with a frequency of a few MHz to 10 MHz.

To carry out a shooting session once, the object 4 is irradiated with the acoustic wave 2, which is a burst wave, at least once. The burst wave has a time waveform in which either a sinusoidal wave or rectangular wave with constant amplitude and frequency (such as a series of the same sinusoidal waves) lasts for a certain period of time. Although not shown in FIG. 1, the time when the acoustic wave source 1 emits the acoustic wave is precisely controlled by a trigger circuit. More specifically, the timing of emission of the acoustic wave 2 and the timing of image capturing by the image receiving section 17 are controlled at a precision on the order of several ns (where 1 ns=10⁻⁹ seconds). For example, the time of transmission from the acoustic wave source 1 and/or the time of shooting by the image receiving section 17 are/is controlled so that the scattered wave 5 based on the acoustic wave 2 propagates as a plane acoustic wave 9 through the acousto-optic medium portion 8 and that the image receiving section 17 captures an image when the plane acoustic wave 9 reaches a plane including the intersection between the acoustic axis 7 and the optical axis 13.

The acoustic wave 2 is mostly a plane wave. Also, a region of the object 4 to be shot is irradiated with the acoustic wave 2 at substantially a uniform intensity. To irradiate the object 4 at substantially a uniform intensity, the acoustic wave 2 may have a larger beam cross section than the image capturing area of the acousto-optic vibrometer 100.

When the object 4 is irradiated with the acoustic wave 2, the acoustic wave 2 is reflected or diffracted inside or from the surface of the object 4, thus creating a scattered wave 5 having the same frequency as the acoustic wave 2. The scattered wave 5 is also a burst wave. The scattered wave 5 has a time waveform in which burst waves created in respective parts of the object 4 are superposed one upon the other. That is why if the acoustic pressure is measured at one point in the medium 3, the acoustic pressure will be obtained as a time waveform in which a lot of burst waves with different amplitudes and timings are superposed one upon the other.

(2) Acoustic Lens System 6

The acoustic lens system 6 transforms the scattered wave 5 into the plane acoustic wave 9 to propagate through the acousto-optic medium portion 8. The acoustic lens system 6 converges the scattered wave 5 by getting a longitudinal wave (compressional wave), which is created when the acoustic wave propagates through the medium, reflected or refracted from/through the interface between two media with different sonic velocities just as light is converged by an optical element in the field of optics. Thus, in the following description, the acoustic lens system 6 will be sometimes described using terms in the field of optics.

In the acousto-optic vibrometer 100, the acoustic lens system 6 functions as an element which converges the scattered waves 5, which have been created at respective points on the focal plane 21, into a predetermined state and which places those scattered waves into a wave in which multiple plane waves with mutually different propagation directions are superposed one upon the other. Hereinafter, a detailed configuration of this acoustic lens system 6 will be described.

The acoustic lens system 6 has a focal length f in the medium 3. The acoustic lens system 6 may be either a refracting acoustic system or a reflecting acoustic system. If the acoustic lens system 6 is a refracting acoustic system, the acoustic lens system 6 includes an acoustic lens which has at least one refracting face and in which the scattered wave 5 is transmitted. The acoustic lens is suitably made of a nanoporous silica, water, a fluorine-based inactive liquid such as fluorinert, polystyrene, or any other elastic body which will cause little acoustic wave transmission loss. The acoustic wave will be refracted by the refracting surface according to the Snell laws of refraction. That is to say, the scattered wave 5 will be refracted at an angle which is determined by the ratio of the sonic velocities of the waves 5 scattered from the materials of the medium 3 and the acoustic lens. On the other hand, if the acoustic lens system 6 is a reflecting acoustic system, then the acoustic lens system 6 will have at least one reflective face which is made of a metal, glass or any other material, of which the acoustic impedance is significantly different from that of the medium 3. These refractive and reflective faces have the same shape as an optical lens or a reflecting mirror, and therefore, can converge the scattered wave 5.

Optionally, an antireflection film having the same function as an antireflection film to be stacked in the field of optics in order to reduce reflection, attenuation and stray light to be produced at a lens refracting surface may be provided for the refracting surface of the acoustic lens system 6. For example, a thin film made of an elastic body, of which the acoustic impedance is equal to the geometric mean of the acoustic impedances of the medium 3 and the acoustic lens and of which the thickness is a quarter wavelength (where the wavelength refers to the wavelength at the frequency of a sinusoidal wave that forms the acoustic wave 2), may be provided as antireflection film for the refracting surface that contacts with the medium 3 of the acoustic lens.

The object 4 is suitably located in the vicinity of the focal plane 21 of the acoustic lens system 6. As described above, as in an optical image capture device such as an optical camera, the more distant from the focal plane 21 of the acoustic lens system 6, the more blurred the real image 18 of the object 4 gets. In this description, the focal plane 21 refers herein to a plane which intersects with the acoustic axis 7 at right angles and which is located closer to the object 4 than the acoustic lens system 6 is by the focal length f of the acoustic lens system 6.

To obtain a sharp real image 18 of the object 4 which is located outside of the focal plane 21, the entire acousto-optic vibrometer 100 is suitably moved so that the object 4 is located in the vicinity of the focal plane 21 of the acoustic lens system 6. If it is difficult to move the acousto-optic vibrometer 100 along the acoustic axis 7 of the acoustic lens system 6 as in an ultrasonic diagnostic apparatus, then the acoustic lens system 6 may further include a focus adjusting mechanism just like an image capturing lens for an optical camera. Furthermore, if the size of the real image 18 needs to be adjustable with respect to the object 4, a focal length adjusting function (i.e., a zoom function) may be provided for one or both of the acoustic lens system 6 and the imaging lens system 16.

To simplify the discussion, it will be described what functions the acoustic lens system 6 has in a situation where the object 4 is located in the vicinity of the focal plane 21. Since the scattered wave 5 is a spherical wave which is centered around an arbitrary point on the focal plane, the spherical wave is transformed by the acoustic lens system 6 into an acoustic wave to propagate through the acousto-optic medium portion 8 with a plane wavefront.

As the spherical wave coming from each point on the focal plane 21 is transformed by the acoustic lens system 6 into such a plane acoustic wave, the plane acoustic wave 9 in the acousto-optic medium portion 8 will be a plane acoustic wave in which a plurality of plane acoustic waves with various traveling directions are superposed one upon the other. Suppose spherical waves are created, on the focal plane 21, from a point A on the acoustic axis 7 of the acoustic lens system 6 and another point B which is located at a distance h from the acoustic axis 7 as shown in FIG. 2. The spherical wave created at the point A is transformed by the acoustic lens system 6 into a plane wave with a plane wavefront A. Since the point A is located on the acoustic axis 7, a normal to the wavefront A becomes parallel to the acoustic axis 7. On the other hand, the spherical wave created at the point B is also transformed into a plane wave with a plane wavefront B. However, a normal to the wavefront B defines an angle φ with respect to the acoustic axis 7. As shown in FIG. 2, the angle φ is equal to Arctan (h/f), where Arctan indicates an arctangent function. Actually, however, a spherical wave is also created from every point between the points A and B. Consequently, the plane acoustic wave 9 shown in FIG. 1 becomes an acoustic wave in which a great number of plane waves, where normals to their wavefront define various angles ψ with respect to the acoustic axis 7, are superposed one upon the other. Also, as will be described in detail later, if the object 4 is moving parallel to the acoustic axis 7, a Doppler shift has been caused by the movement in the frequency of the plane acoustic wave 9.

(3) Acousto-Optic Medium Portion 8

The acousto-optic medium portion 8 is made of an isotropic elastic body which causes little propagation attenuation with respect to the plane acoustic wave 9 and which transmits the sensing light beam 14 to be described later. To improve the image quality (particularly, the resolution) of the real image 18, the speed of sound of the isotropic elastic body that makes the acousto-optic medium portion 8 is suitably as low as possible. Examples of suitable substances with such features include a nanoporous silica, a fluorine-based solvent such as fluorinert, and water.

The acousto-optic medium portion 8 is suitably arranged with respect to the acoustic lens system 6 so that the plane acoustic wave 9 transformed by the acoustic lens system 6 enters the acousto-optic medium portion 8 at a low loss. And the acoustic lens system 6 may be joined with the acousto-optic medium portion 8. Furthermore, to suppress attenuation that would be caused on the junction due to reflection, the junction may also be coated with an antireflection film. If the acoustic lens system 6 and the acousto-optic medium portion 8 are made of the same material, then the acoustic lens system 6 may be provided for a part of the acousto-optic medium portion 8 (which is suitably the boundary with the medium 3). In that case, the acoustic lens system 6 will be formed by one refracting surface.

(4) Acoustic Wave Absorbing End 10

The acousto-optic vibrometer 100 may include an acoustic wave absorbing end 10. The acoustic wave absorbing end 10 is arranged at the other end of the acousto-optic medium portion 8 opposite from its end face with the acoustic lens system 6 and absorbs the plane acoustic wave 9 that has propagated without reflecting or scattering it Since the acoustic wave absorbing end 10 absorbs every acoustic wave that has ever reached the acoustic wave absorbing end 10, only the plane acoustic wave 9 propagates through the acousto-optic medium portion 8. On the other hand, acoustic waves other than the plane acoustic wave 9 will be superposed as images that have nothing to do with the spatial distribution of moduli of elasticity of the object 4, i.e., as noise, on the real image 18. That is why the acoustic wave absorbing end 10 functions as an element which reduces such noise. The material of the acoustic wave absorbing end 10 suitably has approximately the same acoustic impedance as the acousto-optic medium portion 8 and suitably causes significant propagation attenuation to the plane acoustic wave 9 in order to suppress creation of reflected wave at the interface with the acousto-optic medium portion 8. Examples of such materials include rubber and urethane.

Unless the acoustic wave absorbing end 10 is used, an acousto-optic medium portion 8 which is sufficiently long along the acoustic axis 7 may be used, for example. In that case, as the plane acoustic wave 9 propagates through the acousto-optic medium portion 8, the plane acoustic wave 9 attenuates and the reflected wave created at the end can be reduced.

(5) Sensing Light Source 19

The sensing light source 19 generates a sensing light beam 14 in which a great number of plane-wave light beams with mutually different traveling directions are superposed one upon the other. The sensing light beam 14 is incident on the acousto-optic medium portion 8 at an angle with respect to, and neither perpendicularly nor parallel to, the acoustic axis 7 of the acoustic lens system 6. The respective plane-wave light beams yet to be superposed are plane waves and have a high degree of coherence. In this description, the “high degree of coherence” refers herein to a state where wavelengths, traveling directions and phases have all been matched to each other.

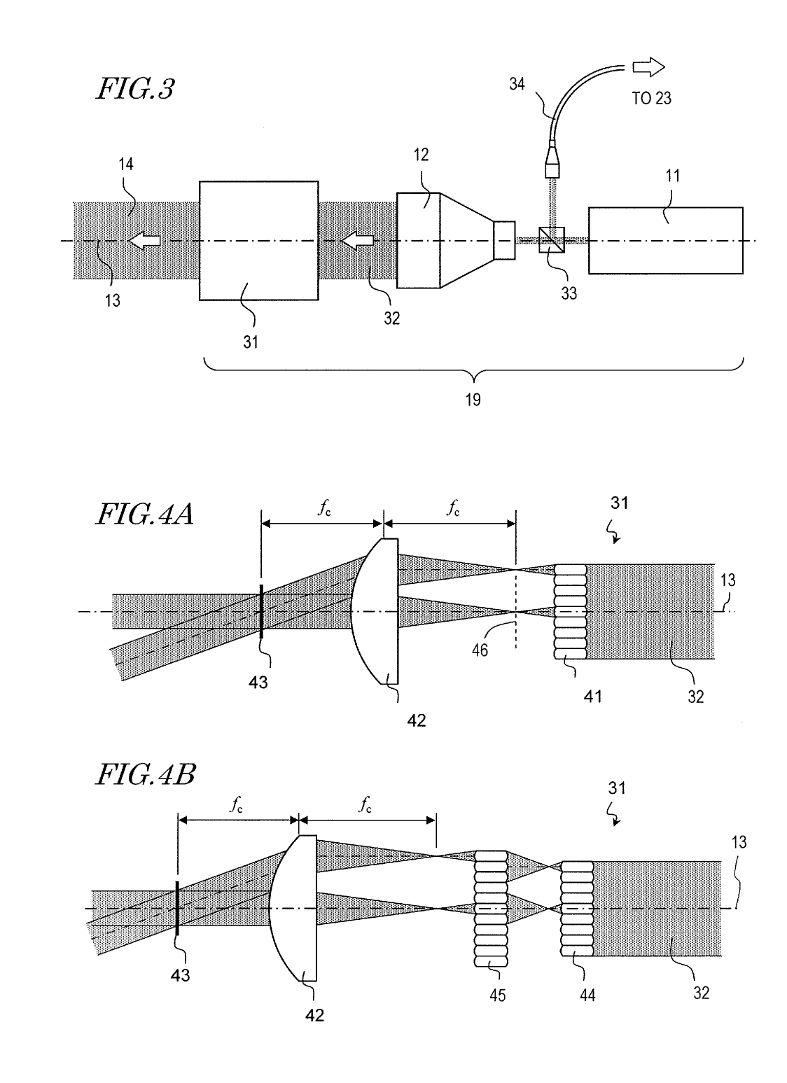

A specific configuration for the sensing light source 19 will be described with reference to FIG. 3. The sensing light source 19 may include a monochromatic light source 11, a beam expander 12 and a uniform illumination optical system 31 as its major components, for example.

The monochromatic light source 11 produces a highly coherent light beam. The rays of light that form the light beam (i.e., the bundle of rays) have the same wavelength and the same phase. Specifically, the light beam emitted from the monochromatic light source 11 suitably has a spectrum width (a half width) of less than 10 nm. Also, the light beam emitted from the monochromatic light source 11 is a plane wave, of which the wavefront accuracy is ten times or less of the wavelength at the center frequency.

As the monochromatic light source 11, a gas laser such as an He—Ne laser, a solid-state laser, or a semiconductor laser, of which the band width has been narrowed by an external resonator, may be used. The light beam emitted from the monochromatic light source 11 may be either a continuous one or a pulsed one, of which the time of emittance is controllable. If the wavelength of the light beam emitted is set to fall within a wavelength range that will cause little transmission loss to the acousto-optic medium portion 8, a high-luminance real image 18 can be obtained. For example, if a nanoporous silica is used as the acousto-optic medium portion 8, a laser having a wavelength of 600 nm or more is suitably used. Then, a high-luminance real image 18 can be obtained.

In this embodiment, the light beam emitted from the monochromatic light source 11 is split by a beam splitter 33 into two light beams. And one of these two light beams is coupled to a single-mode optical fiber 34 and guided to a reference light source 23. The light beam can be coupled to the single-mode optical fiber 34 by getting the light beam condensed to around the core of the single-mode optical fiber 34 by a converging optical system such as a condenser lens. In the configuration shown in FIG. 3, the light beam reflected from the beam splitter 33 is coupled to the single-mode optical fiber 34. However, a transmitted light beam may be guided to the reference light source 23, too. Alternatively, one of the two split light beams may be guided to the reference light source 23 using an optical system other than the single-mode optical fiber 34, e.g., a plurality of plane reflecting mirrors.

The beam expander 12 is arranged on the optical axis 13 as the optical element next to the beam splitter 33. The beam expander 12 increases the diameter of the light beam that has been emitted from the monochromatic light source 11 and outputs a plane-wave light beam 32, of which the diameter has been increased. Even though the beam expander 12 increases the diameter, the wavefront state of the light beam is maintained. That is why the light beam that has been transmitted through the beam expander 12 is also a plane wave.

Next, an exemplary specific configuration for the uniform illumination optical system 31 will be described with reference to FIGS. 4A and 4B. As shown in FIG. 4A, the uniform illumination optical system 31 includes fly-eye lenses and a condenser lens 42. The fly-eye lenses 41 are comprised of n small lenses that are arranged two-dimensionally. Each of those small lenses has an optical axis that is parallel to the optical axis 13. Also, the focal point of every one of those small lenses is located on a focal plane 46 which is a plane that intersects with the optical axis 13 at right angles. Optionally, the respective small lenses may also have mutually different aperture shapes, aperture sizes and focal lengths. The condenser lens 42 has a focal length fc. The optical axis of the condenser lens 42 agrees with the optical axis 13. And the condenser lens 42 is arranged at the distance fc from the focal plane 46.

When the plane-wave light beams 32 are incident on the fly-eye lenses 41, spots are formed by the respective small lenses on the focal plane 46. The total number of those spots is n. The light beams that have converged to leave those spots then travel toward the condenser lens 42 as spherical wave light beams which are centered around the spots. Since the focal plane 46 is also the focal plane of the condenser lens 42, those spherical wave light beams are transformed into plane-wave light beams by the condenser lens 42. However, as the respective optical axes of those spherical wave light beams have shifted parallel from the optical axis 13, those plane-wave light beams travel toward a point on the optical axis which is distant from the condenser lens 42 by the focal length fc, i.e., toward the focal point of the condenser lens 42. That is why the n plane-wave light beams, which are as many as the small lenses, are incident at various angles on, and converge toward, the focal point of the condenser lens 42. In the following description, a plane that includes this focal point and that intersects with the optical axis 13 at right angles will be referred to herein as a “uniformly illuminated plane 43”. If the uniformly illuminated plane 43 is illuminated with a lot of plane-wave light beams at multiple different angles, then it means that at an arbitrary point on the uniformly illuminated plane 43, a lot of rays of light have been incident at various different angles. To make the acousto-optic vibrometer 100 produce a real image 18 of high image quality and high luminance, this is a very important point. The reason will be described in detail later when the Bragg diffraction is described.

If the plane-wave light beams need to be superposed one upon the other at an even larger angle of incidence (where the “angle of incidence” refers herein to the angle defined by the traveling direction of the light beam with respect to the optical axis 13) on the uniformly illuminated plane 43, a condenser lens 42 with an even smaller F number (which is obtained by dividing the focal length by the diameter of the lens aperture) is used. If an image of the object 4 needs to be captured in an even broader range, plane acoustic waves which are further tilted with respect to the acoustic axis 7 are created as shown in FIG. 2. In order to produce Bragg diffracted light based on such plane acoustic waves, plane-wave light beams with an even larger angle of incidence need to be used. That is why by using a condenser lens 42 with a smaller F number, an image of the object 4 can be captured in a wide range.

Also, if an even larger number of plane waves with mutually different angles of incidence need to be superposed one upon the other on the uniformly illuminated plane 43, the fly-eye lenses may be arranged in multiple stages as shown in FIG. 4B. Specifically, the exemplary configuration shown in FIG. 4B includes fly-eye lenses 44 and 45 between the condenser lens 42 and the beam expander 12. A light beam formed by one small lens of the fly-eye lenses 44 is split by the fly-eye lenses 45 into three light beams. As a result, three times as large a number of plane-wave light beams as the number of small lenses that form the fly-eye lenses 45 are incident on the uniformly illuminated plane 43 at mutually different angles.

The uniform illumination optical system 31 functions as not only an optical system to produce groups of light beams with different angles of incidence but also an optical system which produces a light beam with a uniform illumination distribution. On a plane that intersects at right angles with the optical axis 13, the light intensity distribution of the plane-wave light beam 32 emitted from the beam expander 12 is roughly a Gaussian distribution which is rotationally symmetric around the optical axis 13.

On the other hand, on the uniformly illuminated plane 43, the light beams that have been incident on and transmitted through the respective small lenses that form the fly-eye lenses 41 are projected after having been magnified. Suppose small lenses with sufficiently small apertures are used to form the fly-eye lenses. In that case, even if the plane-wave light beams 32 have a light intensity distribution, the light beams incident on the respective small lenses have a substantially uniform light intensity distribution because the respective small lenses have small apertures. Since such very small light beams are magnified and superposed one upon the other on the uniformly illuminated plane 43, the sensing light beam 14 emitted from the uniform illumination optical system 31 has a substantially uniform light intensity distribution on the uniformly illuminated plane 43.

It should be noted that the smaller the aperture of the respective small lenses compared to the light beam diameter of the plane-wave light beams 32 and the larger the number of stages of the fly-eye lenses, the flatter the illumination distribution on the uniformly illuminated plane 43. To produce a real image 18 with no unevenness in illuminance, it is very effective to flatten the illuminance distribution.

As shown in FIG. 5, in this acousto-optic vibrometer 100, the respective members are arranged so that the uniformly illuminated plane 43 is located at the intersection between the acoustic axis 7 and the optical axis 13. As a result, in order to generate a high-luminance real image 18 of high image quality in every image capturing area on the object 4, the entire plane acoustic wave 9 can be illuminated with plane-wave light beams with various angles of incidence. As described above, the uniformly illuminated plane 43 is illuminated with plane-wave light beams with various angles of incidence. Since the uniformly illuminated plane 43 is illuminated with all of those plane-wave light beams in the largest area, the entire plane acoustic wave 9 can be illuminated with the sensing light beam 14 with a smaller beam diameter by arranging the uniformly illuminated plane 43 at the intersection between the acoustic axis 7 and the optical axis 13. Consequently, to reduce the size of the sensing light source 19, the uniformly illuminated plane 43 suitably covers the intersection between the acoustic axis 7 and the optical axis 13.

As will be described in detail later, as the plane acoustic wave 9 propagates through the acousto-optic medium portion 8, the density of the acousto-optic medium portion 8 is varied by the sensing light beam 14. By using this density variation, diffracted light 201 is produced based on the Bragg diffraction of the sensing light beam 14.

(6) Image Distortion Compensating Section 15

The diffracted light 201 produced has the intensity distribution of the plane acoustic wave 9, i.e., an intensity distribution representing the two-dimensional distribution of elastic properties of the object 4 on the focal plane 21. However, since the diffracted light 201 is emitted obliquely to the acoustic axis 7 which indicates the traveling direction of the plane acoustic wave 9, the intensity distribution is distorted. Thus, the image distortion compensating section 15 corrects the distortion of the diffracted light 201. Alternatively, the distortion of the diffracted light 201 may also be corrected by the image processing section 20.

(7) Reference Light Source 23

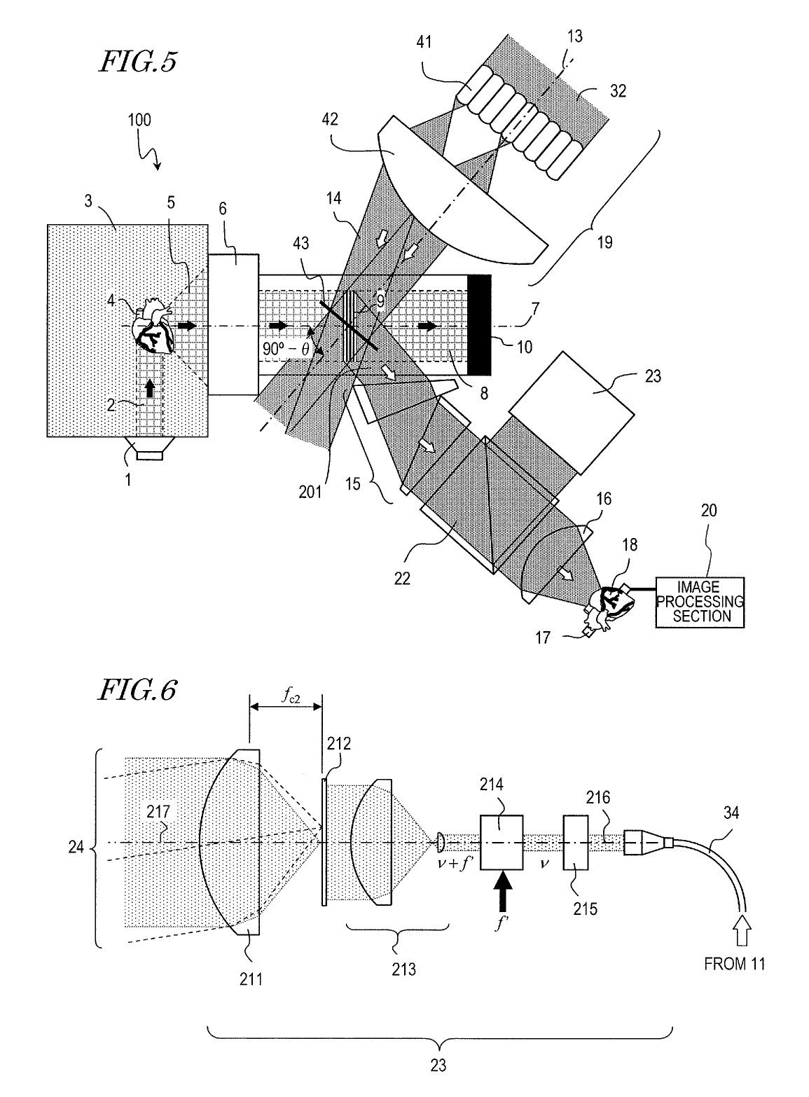

The reference light source 23 emits a reference light beam 24 to be superposed on the diffracted light 302 (or diffracted light 201) based on the sensing light beam 14 that has been produced by the acousto-optic medium portion 8. As shown in FIG. 6, the reference light source 23 includes a shutter 215, an acousto-optic modulator 214, a beam expander 213, a light scattering plate 212 and a condenser lens 211. The beam splitter 22 may be used to superpose the diffracted light 302 (or diffracted light 201) and the reference light beam 24 one upon the other.

In this embodiment, the parallel light beam 216 is obtained by splitting the light beam that has been emitted from the monochromatic light source 11 of the sensing light source 19 and guiding it to the single-mode optical fiber 34. The monochromatic light guided is transformed into a plane-wave light beam by a condenser lens (not shown), of which the focal point is located at the core end face of the single-mode optical fiber 34. That is why the parallel light beam 216 is a highly coherent plane-wave light beam that has the same frequency as the monochromatic light emitted from the monochromatic light source 11.

The parallel light beam 216 passes through the shutter 215 which performs switching on the light beam, and then enters the acousto-optic modulator 214, which is an optical element that changes the frequency of the monochromatic light included in the parallel light beam 216 (i.e., makes frequency modulation). More specifically, if the frequency of the parallel light beam 216 yet to enter the acousto-optic modulator 214 is ν and if the frequency of a sinusoidal wave signal to be supplied to the acousto-optic modulator 214 is f′, then the light beam to be output from the acousto-optic modulator 214 has a frequency ν+f′. As the acousto-optic modulator 214, an optical element which subjects the light beam to the Bragg diffraction by using the variation in the density of the acoustic propagation medium to be cause when the acoustic wave propagates through the acoustic propagation medium may be used, for example. As the acoustic propagation medium, tellurium dioxide may be used, for example.

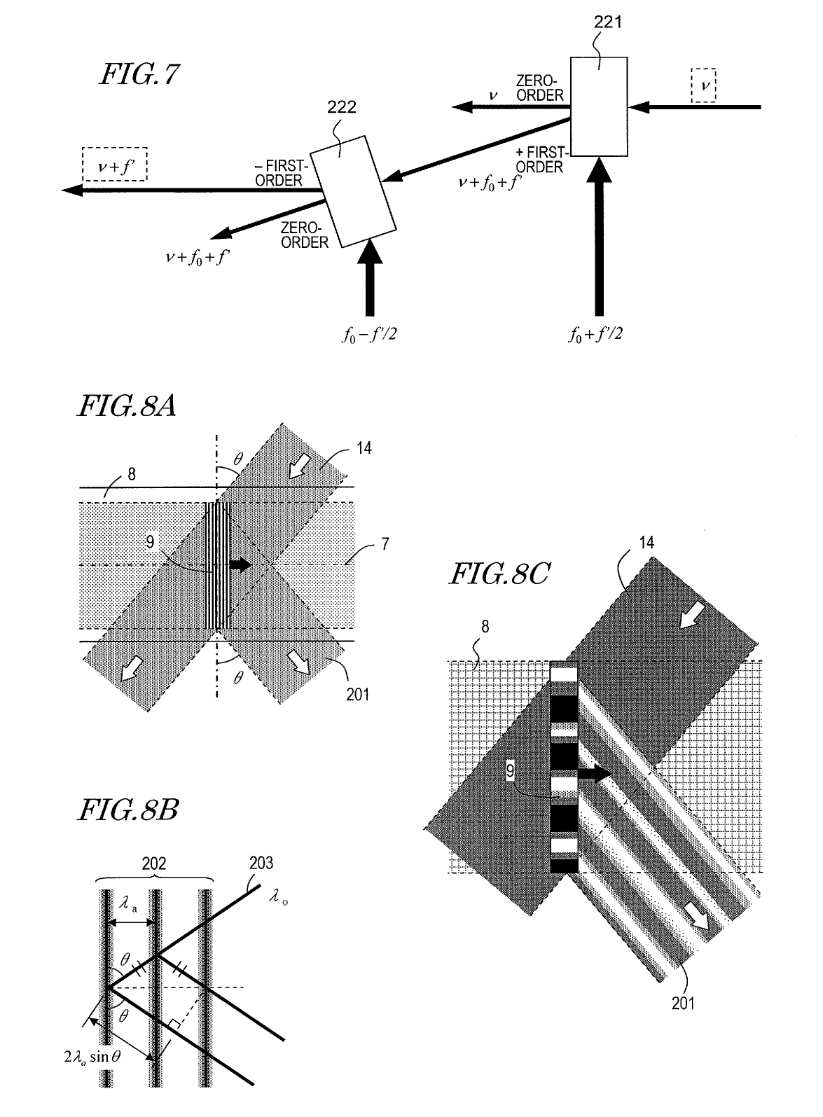

The light intensity of the parallel light beam 216 that has just been subjected to the frequency modulation and has gone out of the acousto-optic modulator 214 generally depends heavily on the frequency f′ of the sinusoidal wave signal to be supplied to the acousto-optic modulator 214. To generate a high-intensity frequency-modulated parallel light beam using a sinusoidal wave signal with an arbitrary frequency, the reference light source 23 may include plurality of acousto-optic modulators as shown in FIG. 7, for example. Specifically, the reference light source 23 may include a first acousto-optic modulator 221 and a second acousto-optic modulator 222. When the frequency of the incoming sinusoidal wave signal is f₀, the first and second acousto-optic modulators 221 and 222 obtain the maximum diffraction efficiency (and when the diffraction efficiency becomes maximum, the light intensity of the frequency-modulated light beam also becomes maximum). Signals with frequencies f₀+f′/2 and f₀−f′/2 are supplied to the first and second acousto-optic modulators 221 and 222, respectively, and the first and second acousto-optic modulators 221 and 222 are arranged so that the +first-order diffracted light that has gone out of the first acousto-optic modulator 221 enters the second acousto-optic modulator 222. For example, if the movement of the vascular wall is observed with an acoustic wave 2, of which the frequency f falls within the range of approximately 3 to 10 MHz, f′ is almost as high as f. If tellurium dioxide is used as the acoustic propagation medium for the first and second acousto-optic modulators 221 and 222, f₀ falls within the range of approximately 50 to 150 MHz. It should be noted that to observe a slower movement with high precision, f′ is selected so that |f′−f| becomes about 1 kHz.

In this case, the −first-order diffracted light to go out of the second acousto-optic modulator 222 becomes a light beam that has been subjected to the frequency modulation at the frequency ν+f′. In this manner, a high-intensity frequency-modulated parallel light beam 216 with the frequency ν+f′ can be obtained. It should be noted that even if the first and second acousto-optic modulators 221 and 222 are arranged in reverse order, a similar frequency-modulated parallel light beam 216 can also be obtained. Furthermore, even if the frequencies of the signals supplied to the first and second acousto-optic modulators 221 and 222 are f₀+Δ₁f and f₀−Δ₂f (where Δ₁f>0, Δ₂f>0 and Δ₁f+Δ₂f=f′), respectively, a high-intensity parallel light beam 216 having the frequency ν+f′ can also be generated.

As shown in FIG. 6, the beam expander 213 transforms the parallel light beam 216, of which the frequency has been modulated into ν+f′, into a light beam with a large beam cross-sectional diameter, and irradiates the light scattering plate 212 with such a light beam. The light beam with such a large beam cross-sectional diameter does not have to be a plane wave. For example, the beam expander 213 may be replaced with either a single convex lens or a single concave lens. Nevertheless, the expanded light beam suitably has an approximately uniform illuminance distribution on the beam cross section.

Frosted glass may be used as the light scattering plate 212. In that case, the surface roughness of the frosted glass is suitably as small as possible for the following two reasons. Firstly, the scattered light produced by the light scattering plate 212 would have a high light intensity in the direction parallel to the optical axis 217. Then, a good reference light beam 24 can also be generated based on even a parallel light beam 216 with a lower intensity. In addition, the light intensity distribution of the reference light beam 24 that has been transmitted through the light scattering plate 212 on a plane that intersects at right angles with the optical axis 217 would be reflected on the real image of the object 4 to be sensed by the image receiving section 17 and the real image would include a speckle pattern. In this description, the “speckle pattern” refers herein to a two-dimensional optical image in which luminous points and dark points are randomly distributed. The scattered rays of light produced from very small points that form the surface morphology of the light scattering plate 212 are superposed one upon the other and interfere with each other, thus generating a speckle. Optionally, the light scattering plate 212 may be replaced with a uniform illumination optical system which is implemented as the fly-eye lens shown in FIG. 4A. If such a uniform optical system implemented as a fly-eye lens is used, it is possible to prevent the speckle from being generated.

If the surface roughness of the frosted glass is greater than the wavelength of the light beam emitted from the monochromatic light source 11, the sizes of the luminous and dark points are determined mostly by the composite focal length of the condenser lens 211 and the imaging lens system 16 and by the aperture size of the condenser lens 211. The smaller the value obtained by dividing the composite focal length by the aperture size of the condenser lens 211, the smaller the sizes of the luminous and dark points are. To allow this acousto-optic vibrometer 100 to increase the measuring resolution of the displacement velocity distribution on the object 4, the sizes of the luminous and dark points of the speckle pattern on the photosensitive plane of the image receiving section 17 are suitably at least smaller than the resolution of the image of the acoustic wave on the photosensitive plane of the image receiving section 17. That is why the sizes of the luminous and dark points on the photosensitive plane of the image receiving section 17 are suitably reduced and a condenser lens 211 with a larger aperture size and a shorter focal length f_{c2} is suitably used. Supposing the composite focal length is l, the aperture size is d, and the wavelength of the light emitted from the monochromatic light source 11 is λ, the sizes Δ of the luminous and dark points on the photosensitive plane of the image receiving section 17 is 1.22×λ1/d. That is why the aperture size d and focal length f_{c2} of the condenser lens 211 are determined so that Δ becomes equal to or smaller than the measuring resolution.

From the respective points on the light scattering plate 212 which is illuminated with the parallel light beam 216, of which the frequency has been modulated into ν+f′, produced are scattered rays of light having the frequency ν+f′. In the reference light source 23 shown in FIG. 6, the condenser lens 211 having the focal length f_{c2} is arranged at the distance f_{c2} from the light scattering plate 212, thereby transforming the scattered rays of light coming from the light scattering plate 212 into a plane-wave light beam. Since the scattered rays of light are produced from the respective points on the light scattering plate 212, the traveling direction of the plane-wave light beam going out of the condenser lens 211 is not parallel to the optical axis 217 of the condenser lens 211. And its angle depends on where on the light scattering plate 212 the scattered ray of light has been produced. That is why in the light beam going out of the condenser lens 211, a lot of plane-wave light beams consisting of monochromatic rays of light having the frequency ν+f′ and mutually different traveling directions are superposed one upon the other. Since a great number of plane-wave light beams consisting of monochromatic rays of light with mutually different traveling directions are superposed one upon the other, the reference light beam 24 emitted from the reference light source 23 is similar to the sensing light beam 14 produced from the sensing light source 19.

The reference light source 23 shown in FIG. 6 includes the shutter 215 to turn ON and OFF (i.e., selectively pass) the reference light beam 24. However, instead of using the shutter 215, the sinusoidal wave signal supplied to the acousto-optic modulator 214 may be turned ON and OFF. In that case, while the sinusoidal wave signal is being supplied to the acousto-optic modulator 214, the reference light source 23 emits a reference light beam 24 having the frequency ν+f′. On the other hand, while no sinusoidal wave signal is supplied, the reference light source 23 outputs a light beam having the frequency ν.

(8) Imaging Lens System 16 and Image Receiving Section 17

The imaging lens system 16 condenses the diffracted light 302, on which the reference light beam 24 is superposed, onto the photosensitive plane of the image receiving section 17. The image receiving section 17 includes a plurality of pixels (photoelectric transducers) which are arranged two-dimensionally, detects the condensed diffracted light 302 two-dimensionally and outputs an electrical signal. The electrical signal thus generated represents the two-dimensional distribution and displacement velocity distribution of the elastic properties of the object 4 on the focal plane 21. And by analyzing the electrical signal thus obtained, an image representing the two-dimensional distribution of the elastic properties or displacement velocities of the object can be obtained.

2. Operation of Acousto-Optic Vibrometer 100

Next, it will be described how this acousto-optic vibrometer 100 operates. The acousto-optic vibrometer 100 of this embodiment obtains an image of the object 4 based on an acoustic wave (i.e., an image representing the distribution of elastic properties) and an image for use to measure the shift velocity distribution at respective parts of the object 4. Hereinafter, it will be described how to obtain these two images.

(1) Operation when Acoustic Wave Image is Obtained

First of all, it will be described how the acousto-optic vibrometer 100 obtains an acoustic wave image. As described above, the sensing light beam 14 is comprised of a lot of plane-wave light beams with mutually different traveling directions, and the plane acoustic wave 9 is also comprised of a lot of plane acoustic waves with mutually different traveling directions. However, in the following description, the sensing light beam 14 is supposed to consist of only a single plane-wave light beam, of which the wavefront intersects at right angles with the optical axis 13, and the plane acoustic wave 9 is supposed to consist of only a single plane acoustic wave which intersects at right angles with the acoustic axis 7 for the sake of simplicity.

As shown in FIG. 1, the sensing light beam 14 is incident obliquely to the acousto-optic medium portion 8 so that the light beam 14 becomes neither perpendicular nor parallel to the acoustic axis 7 of the acoustic lens system 6. The angle formed between the acoustic axis 7 and the optical axis 13 of the sensing light beam 14 is calculated by 90 degrees minus θ. That is to say, θ is the angle of incidence of the sensing light beam 14 on the wavefront of the plane acoustic wave 9. As described above, the angle θ may be any arbitrary angle other than 0, 90, 180 and 270 degrees as long as the light beam 14 is neither perpendicular nor parallel to the acoustic axis 7. Only at θ falling within this angular range, Bragg diffraction is caused in the sensing light beam 14, thus producing diffracted light 201. A specific method for setting that angle θ at which the diffracted light 201 is produced will be described later.

As described above, in this acousto-optic vibrometer 100, the time of emission of the acoustic wave 2 is controlled so precisely that the plane acoustic wave 9 will have reached the uniformly illuminated plane 43 exactly by the time to shoot for the image receiving section 17. Specifically, if the interval of emission of the acoustic wave 2 is controlled at a time precision of 1 ns, the plane acoustic wave 9 propagating through the acousto-optic medium portion 8 at a speed of sound of 50 m/s will have a positional error of 50 nm. If an He—Ne laser is used as the monochromatic light source 11, this positional error corresponds to 0.079 wavelength when converted into 633 nm which is the wavelength of the He—Ne laser. That is why it can be seen that by adjusting the time of emission of the acoustic wave 2, the position of the plane acoustic wave 9 in the acousto-optic medium portion 8 can be controlled highly precisely.

FIG. 8A illustrates how the sensing light beam 14 is subjected to the Bragg diffraction by the plane acoustic wave 9 in a situation where the respective positions of the sensing light beam 14 and the plane acoustic wave 9 are controlled as described above. FIG. 8A schematically illustrates the moment when the plane acoustic wave 9 crosses the optical path of the sensing light beam 14. The plane acoustic wave 9 is a compressional wave propagating through the acousto-optic medium portion 8. That is why a refractive index distribution which is proportional to the acoustic pressure distribution in the plane acoustic wave 9 is formed in the acousto-optic medium portion 8. Since the plane acoustic wave 9 is a sinusoidal wave with a single frequency as described above, the refractive index distribution formed comes to have a periodic structure in which one period parallel to the acoustic axis 7 is equal to the wavelength of the plane acoustic wave 9 and in which the magnitude of the refractive index changes in a sinusoidal wave but is uniform perpendicularly to the acoustic axis 7. Such a periodic refractive index distribution functions as a one-dimensional diffraction grating with respect to the sensing light beam 14. That is why if the sensing light beam 14 is incident on the plane acoustic wave 9 at an angle θ that satisfies the diffraction condition to be described below, the diffracted light 201 is produced. Since this one-dimensional diffraction grating has a planar lattice plane and since the wavefront of the sensing light beam 14 is also plane, the diffracted light 201 becomes a plane-wave light beam.

In the acousto-optic vibrometer 100 of the present invention, the acoustic wave 2 is comprised of sinusoidal waves, of which the number of periods is far larger than two, and therefore, the one-dimensional diffraction grating with a large number of lattice planes operates as an amplitude type phase lattice and the diffraction produced there is the Bragg diffraction. According to Bragg diffraction, the angles defined by the sensing light beam 14 and the diffracted light 201 with respect to the plane acoustic wave 9 are equal to each other and are both θ as shown in FIG. 8A. The angle θ is a discrete value that satisfies the Bragg diffraction condition to be described later. If the acoustic wave 2 is comprised of a small number of sinusoidal waves, of which the number of periods is as small as two, and if the acoustic wave 2 functions as a phase type diffraction grating, the diffracted light 201 is produced mainly by Raman-Nath diffraction. According to a pure Raman-Nath diffraction, the angles defined by the sensing light beam 204 and the diffracted light 201 with respect to the plane acoustic wave 9 do not have to be equal to each other. The Bragg diffraction will produce diffracted light 201 with a higher intensity than the Raman-Nath diffraction will. That is why according to Bragg diffraction, scattered wave 5 with a lower acoustic pressure can be observed effectively. For that reason, in the acousto-optic vibrometer 100 of the present invention, diffracted light 201 produced mainly by Bragg diffraction is adopted using the acoustic wave 2 comprised of a large number of sinusoidal waves. In capturing an image actually, an acoustic wave 2 comprised of less than ten sinusoidal waves is used, and therefore, the diffracted light 201 includes Raman-Nath diffracted light. However, as will be described later, such an inclusion of the Raman-Nath diffracted light into the diffracted light 201 works fine to produce a good real image 18.

The Bragg diffraction condition to be imposed on a one-dimensional diffraction grating formed by the plane acoustic wave 9 will be described. FIG. 8B schematically shows the Bragg diffraction condition to be imposed on the one-dimensional diffraction grating formed by the plane acoustic wave 9. As shown in FIG. 8B, the lattice spacing of the diffraction grating 202 formed by the plane acoustic wave 9 is equal to the wavelength λₐ of the acoustic wave propagating through the acousto-optic medium portion 8. In the following description, one monochromatic ray of light in the sensing light beam 14 will be referred to herein as a “monochromatic ray 203”. The wavelength of the monochromatic ray 203 is supposed to be λ₀. If the monochromatic ray 203 is incident on the diffraction grating 202, weak scattered light is produced by each lattice. Looking at rays of light scattered from adjacent lattice planes, if the difference in optical path length (2×λₐ×sin θ) between two rays of light scattered toward the same direction from the respective lattice planes is equal to an integral multiple of the wavelength λ0 (m×λ₀, m=±1, ±2, . . . ), then the two scattered rays of light will enhance each other. Since such an enhancement will be caused on other lattice planes as well, scattered light with an overall high intensity (i.e., diffracted light) is produced. For these reasons, the angle θ at which the diffracted light is observed is represented by the following Equation (1):

$$θ = Arcsin ( \frac{{λ}_{O} / {λ}_{a}}{2 \times m} ) , \\ ( m = \pm 1 , \pm 2 , … ) \qquad ( 1 )$$

This Equation (1) specifies the Bragg diffraction condition and defines the angle θ formed between incoming and outgoing rays of light with respect to a lattice plane. In Equation (1), Arcsin indicates an arctangent function. A pure Bragg diffraction refers herein to a diffraction phenomenon to occur in a situation where the diffraction grating 202 is comprised of an infinite number of lattice planes. As shown in FIG. 8B, the angles defined by incoming and outgoing rays of light with respect to the lattice plane are equal to each other and are both θ. If the diffraction grating formed by the plane acoustic wave 9 is an amplitude type diffraction grating with a sinusoidal amplitude distribution, diffracted light is obtained according to the Bragg diffraction only when the order m=0 or ±1. However, since the Raman-Nath diffracted light is ordinarily included, high-order diffracted light, which satisfies |m|>1, will be produced. If the plane acoustic wave 9 is weak, generally speaking, the smaller the order m of diffracted light 201, the higher its intensity will be. For that reason, to observe a weaker scattered wave 5, diffracted light 201 which satisfies m=±1 is suitably used. Even though the diffracted light satisfies m=±1 in the acousto-optic vibrometer shown in FIG. 1, an acousto-optic vibrometer that uses diffracted light, of which m=−1, may also be used.

Next, it will be described with reference to FIG. 8C how the diffracted light 201 has a light intensity distribution, which is proportional to the acoustic pressure distribution on the wavefront of the plane acoustic wave 9, on its wavefront. As shown in FIG. 8C, the plane acoustic wave 9 generally has a non-uniform acoustic pressure distribution on the wavefront. Since the spatial distribution of the variation in refractive index in the acousto-optic medium portion 8 is proportional to the acoustic pressure distribution of the plane acoustic wave 9, the in-plane distribution of the refractive index variation on a lattice plane of the diffraction grating 202 is non-uniform. Supposing the object 4 has only minimal displacement during the duration of each pulse of the acoustic wave 2 and can be regarded as standing still, the refractive index distribution is the same on every lattice plane of the diffraction grating 202. That is why the diffraction grating 202 becomes a one-dimensional diffraction grating and the diffracted light 201 is produced mainly by Bragg diffraction (but actually some Raman-Nath diffracted light is mixed as described above). In this case, the amplitude of the diffracted light 201 (which is obtained by raising the light intensity to the halfth power) is proportional to the magnitude of the refractive index variation, and therefore, the amplitude of the diffracted light 201 is proportional to the acoustic pressure distribution of the plane acoustic wave 9. Consequently, the light amplitude distribution of the diffracted light 201 on the wavefront is proportional to the acoustic pressure distribution on the plane acoustic wave 9.

The diffracted light 201 leaves the acousto-optic medium portion 8 and enters an image distortion compensating section 15. Hereinafter, it will be described with reference to FIG. 9A how the image distortion compensating section 15 operates. FIG. 9A is a schematic representation showing how the diffracted light 201 converges in one direction in this acousto-optic vibrometer 100. As can be seen from Equation (1), to satisfy the diffraction condition, the sensing light beam 14 needs to be incident obliquely with respect to the plane acoustic wave 9. In this case, the plane acoustic wave 9 is supposed to have a circular beam cross section with a diameter L and the angle of diffraction of the diffracted light 201 is supposed to be θ (which is just as defined in the foregoing description). As described above, the sensing light beam 14 has a beam diameter which is large enough to cover the plane acoustic wave 9 and the diffracted light 201 is produced only in a region where there is the plane acoustic wave 9. That is why the beam cross section of the diffracted light 201 becomes an elliptical one, of which the minor axis size is L×sin θ as measured in the y-axis direction and the major axis size is L as measured in the x-axis direction in the coordinate system shown in FIG. 9A. That is to say, the distribution of the amplitude of the light on the wavefront of the diffracted light 201 is proportional to the acoustic pressure distribution of the plane acoustic wave 9 on the wavefront multiplied by sin θ in the y-axis direction.

That is why if the diffracted light 201 that has converged in one direction were imaged as it is by the imaging lens system 16 to produce a real image 18, then the real image 18 would be an optical image distorted in the y-axis direction and the analogy between the object 4 and the real image 18 would be lost. That is to say, the diffracted light 201 has distortion in the y-axis direction. For that reason, the distortion of the diffracted light 201 is corrected by the image distortion compensating section 15.

In this embodiment, the image distortion compensating section 15 includes an anamorphic prism 301. Hereinafter, the configuration and function of the anamorphic prism 301 will be described with reference to FIG. 9B. FIG. 9B is a schematic representation illustrating the configuration of the anamorphic prism 301. As shown in FIG. 9B, the anamorphic prism 301 includes two wedge prisms 303.

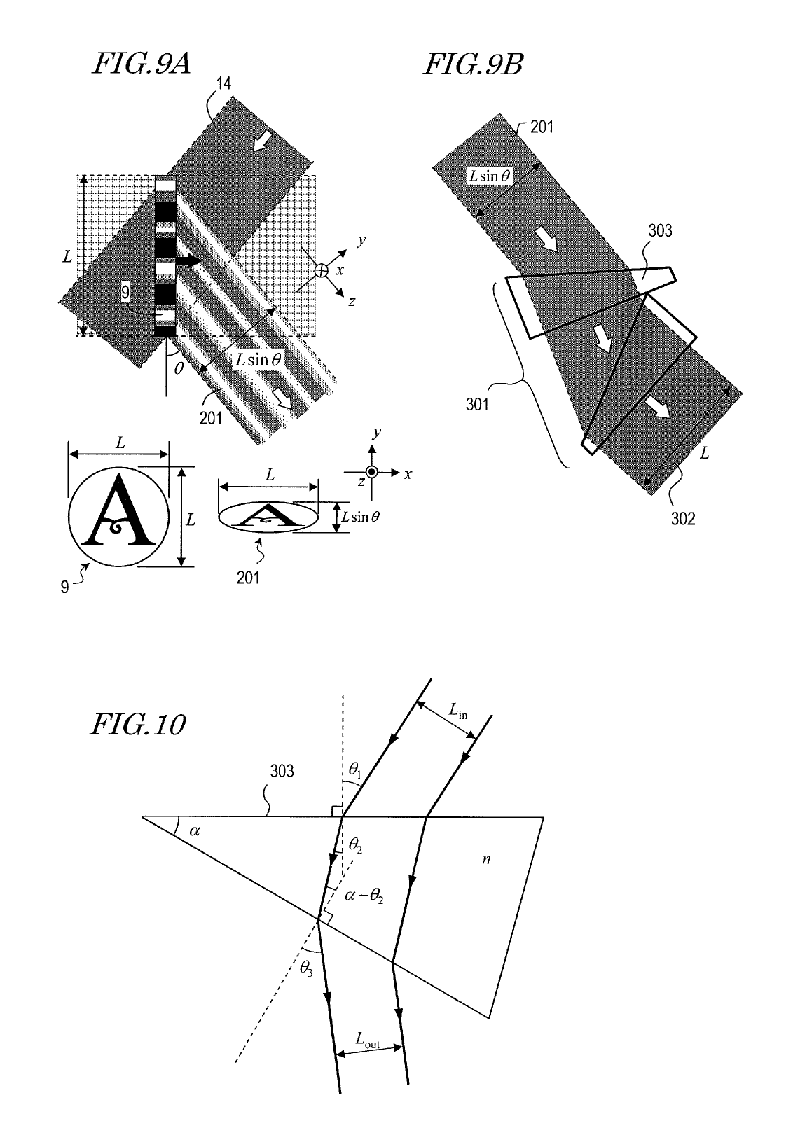

Hereinafter, it will be described with reference to FIG. 10 how these wedge prisms 303 work. FIG. 10 is a ray of light tracking diagram illustrating how rays of light are transmitted through the wedge prism 303. The wedge prism 303 is in a medium with a refractive index of one and is made of a glass material with a refractive index n. The wedge prism 303 has a columnar shape with the cross-sectional shape shown in FIG. 10 and with a uniform thickness. FIG. 10 illustrates a cross section of the wedge prism 303 as viewed on a plane including normals to two planes that interpose an acute angle α between them.

If a light beam which is parallel to such a plane including normals to the two planes that interpose the acute angle α between them is incident on the wedge prism 303, a ray of light which is refracted in a direction that is parallel to the same plane will go out. The angle of incidence of such a light beam on a first refracting plane will be identified herein by θ₁, the angle of emittance from the first refracting plane by θ₂, and the angle of emittance from a second refracting plane by θ₃. Also, the width of the light beam incident on the first refracting plane is supposed to be Lᵢₙ and the width of the light beam going out of the second refracting plane is supposed to be Lₒᵤₜ. In this case, if θ₁, α and n are given, θ₂ and θ₃ can be calculated by the following Equations (2):

sin θ₁=n×sin θ₂

n×sin(α−θ₂)=sin θ₃ (2)

Also, as can be seen from FIG. 10, the incoming light beam and the light beam going out of the wedge prism 303 have mutually different beam diameters. If the signs shown in FIG. 10 are used, the light beam expansion ratio, calculated by Lₒᵤₜ/Lᵢₙ, is given by the following Equation (3):

$$\frac{{L}_{out}}{{L}_{in}} = \sqrt{\frac{{n}^{2} + ( {n}^{2} - 1 ) {tan}^{2} {θ}_{1}}{{n}^{2} + ( {n}^{2} - 1 ) {tan}^{2} {θ}_{3}}} \qquad ( 3 )$$

As can be seen from these Equations (2) and (3), by selecting α, n and angle of incidence θ₁ appropriately for the wedge prism 303, any light beam expansion ratio can be obtained just as intended.

As shown in FIG. 9B, the anamorphic prism 301 is obtained by combining more than one wedge prism 303 shown in FIG. 10. If two wedge prisms 303 of the same shape are used in combination as shown in FIG. 9B, the light incident on the anamorphic prism 301 and the light going out of the anamorphic prism 301 can be made parallel to each other, and the optical system can be adjusted easily.

Based on the principle described above, the anamorphic prism 301 operates as an optical system that expands the diameter of the light beam. In the acousto-optic vibrometer 100, α, n and the angle of incidence θ₁ are selected for the wedge prism 303, and the diffracted light 201 is expanded by the factor of 1/sin θ in the y-axis direction as shown in FIG. 9B. In this manner, distortion-compensated diffracted light 302 with a circular beam cross section having a diameter L can be obtained. Consequently, the distortion-compensated diffracted light 302 has, on its wavefront, a light amplitude distribution which is proportional to the acoustic pressure distribution on the wavefront of the plane acoustic wave 9. That is to say, even though the distortion-compensated diffracted light 302 has a different wavelength from the plane acoustic wave 9 that is an ultrasonic wave, the entire acoustic pressure distribution on the wavefront of the plane acoustic wave 9 is reproduced as a light amplitude distribution. That is why a real image 18 which is similar to the object 4 can be produced with certainty.

Now, it will be further described with reference to FIG. 1 again how the acousto-optic vibrometer 100 obtains an acoustic wave image. As shown in FIG. 1, the reference light beam 24 that has been generated by the reference light source 23 is superposed on the distortion-compensated diffracted light 302 while passing through the beam splitter 22 and then the diffracted light 302 is condensed by the imaging lens system 16 having the focal length F. The diffracted light 302 and the reference light beam 24 are parallel light beams, and therefore, condensed onto the focal plane of the imaging lens system 16 to produce a real image 18 there. In this description, the focal plane of the imaging lens system 16 refers herein to a plane which intersects at right angles with the optical axis of the imaging lens system 16 and which is located at a distance F (which is the focal length of the imaging lens system 16) as measured from the imaging lens system 16 toward the image receiving section 17. That is to say, these members are arranged so that the photosensitive plane of the image receiving section 17 is located at the focal plane of the imaging lens system 16. And the real image 18 on the focal plane is obtained as an optical image.

In the foregoing description, the sensing light beam 14 is supposed to consist of only a plane-wave light beam, of which the wavefront intersects at right angles with the optical axis 13, and the plane acoustic wave 9 is supposed to consist of only a plane acoustic wave which intersects at right angles with the acoustic axis 7. However, since the object 4 is not a point on the acoustic axis 7 but has a finite size as already described with reference to FIG. 2, the plane acoustic wave 9 that has been transformed by the acoustic lens system 6 includes a lot of plane acoustic waves which do not intersect at right angles with the acoustic axis 7. In the acousto-optic image capture device of this embodiment, as the sensing light beam 14 is formed by superposing a plurality of monochromatic rays of light with mutually different traveling directions one upon the other, even those plane acoustic waves 9 with different traveling directions can also produce Bragg diffracted light.

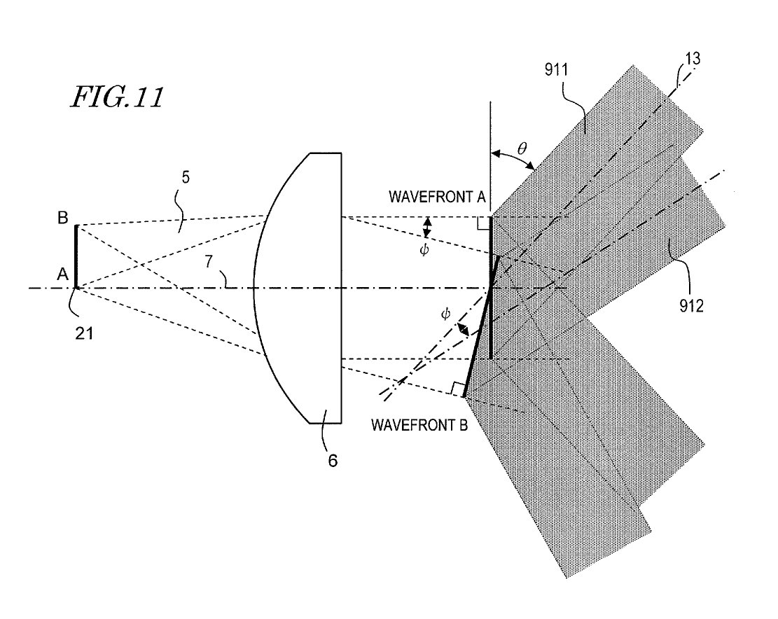

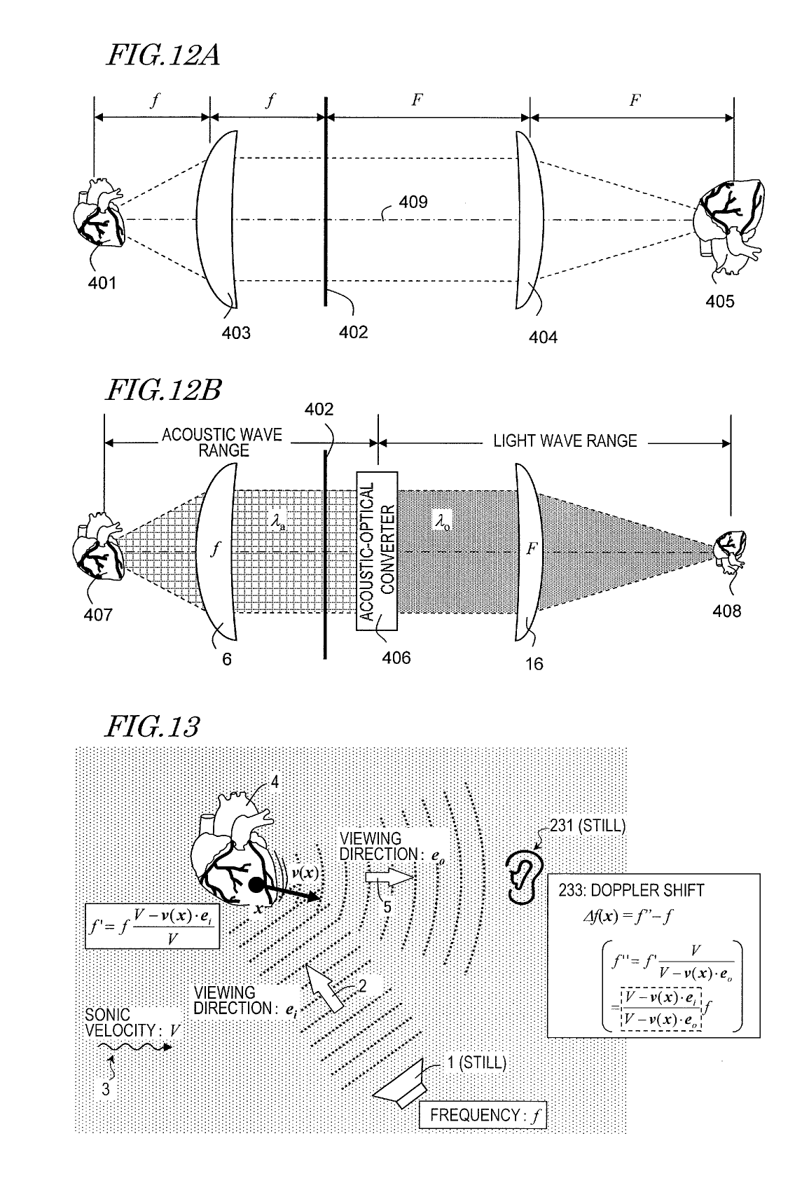

FIG. 11 shows how spherical waves which have been created at two points A and B on the object 4 and on the focal plane 21 of the acoustic lens system 6 are transformed into a plane acoustic wave to produce Bragg diffracted light. Although the point A is located at the intersection between the acoustic axis 7 and the focal plane 21 as in FIG. 2, the point B is not located on the acoustic axis 7. As shown in FIG. 11, the wavefront A of the plane acoustic wave based on the scattered wave 5 that has been created at the point A is a plane which intersects at right angles with the acoustic axis 7. However, the wavefront B of the plane acoustic wave based on the scattered wave 5 that has been created at the point B, which is off the acoustic axis 7, is not a plane which intersects at right angles with the acoustic axis 7 but defines an angle ψ with respect to the acoustic axis 7. In this case, the angle ψ is just as defined above with reference to FIG. 2.

Now look at a plane-wave light beam 911 which is parallel to the optical axis 13 among a lot of plane-wave light beams that have been produced by the sensing light source 19. The angle formed between the acoustic axis 7 and the optical axis 13 is adjusted so that the plane-wave light beam 911 is incident on the wavefront A at such an angle θ that satisfies the Bragg diffraction condition. That is why diffracted light is produced at the wavefront A. On the other hand, the angle of incidence of the plane-wave light beam 911 with respect to the wavefront B becomes θ−ψ, and therefore, the Bragg diffraction condition is not satisfied and no diffracted light is produced. Consequently, with only the plane-wave light beam 911 used, no diffracted light will be produced based on the scattered wave 5 that has been created at the point B and an optical image corresponding to the point B will be missing from the real image 18.

To produce diffracted light at the wavefront B, the wavefront B may be irradiated with a plane-wave light beam 912 which defines a tilt angle ψ clockwise with respect to the optical axis 13 as shown in FIG. 11. Since the plane-wave light beam 912 is incident on the wavefront B at an angle θ, diffracted light corresponding to the plane acoustic wave 9 that has been created at the point B will be produced. In that case, an optical image corresponding to the point B will not be missing from the real image 18.

As can be seen, to get optical images corresponding to the points A and B included in the real image 18, both of the plane-wave light beams 911 and 912 are used. Likewise, to get optical images corresponding to points other than the points A and B included in the real image 18, a plane-wave light beam, of which the angle of incidence is defined so as to make diffracted light corresponding to the scattered wave 5 created at those points appear, is used. That is why by using the sensing light beam 14 in which a plurality of monochromatic rays of light with mutually different traveling directions are superposed one upon the other, not only a region on the acoustic axis 7 but also regions surrounding the acoustic axis 7 can be shot on the focal plane 21. As a result, an acousto-optic vibrometer 100 which can shoot the object 4 at a wide viewing angle is realized. In addition, the shooting session for shooting the object 4 can get done by detecting the diffracted light as an optical image without performing any complicated signal processing such as delay synthetic signal processing in a conventional ultrasonic diagnostic apparatus. Consequently, an image of the object 4 can be obtained at high speeds.

On the focal plane 21, the actual object 4 is comprised of an infinite number of points. That is why to shoot the object 4 with high resolution, an infinite number of plane-wave light beams should be prepared. And if only a finite number of plane-wave light beams with discrete angles of incidence are used as is done in this embodiment, the real image 18 seems to be an optical image consisting of as many discrete points as the number of the plane-wave light beams. However, the plane acoustic wave 9 is a pulsed acoustic wave and is comprised of a finite number of wavefronts. That is why the number of lattice planes of the diffraction grating to be formed in the acousto-optic medium portion 8 becomes a finite one, too. As described above, the diffracted light produced by a diffraction grating with a finite number of lattice planes includes not only Bragg diffracted light but also Raman-Nath diffracted light. The diffraction condition of the Raman-Nath diffraction does not depend on the angle of incidence. For that reason, even if the object is irradiated with only the plane-wave light beam 911, actually not only an optical image at the point A but also optical images in the vicinity of the point A will be generated as parts of the real image 18. Consequently, the real image 18 to generate is not a set of discrete points but actually becomes a continuous optical image which is similar to the object 4.

Each of the plane-wave light beams to be superposed by the sensing light source 19 actually has a finite beam diameter. If each parallel light beam has a finite beam diameter, then it means that “perfect” plane waves with various traveling directions have been superposed one upon the other in those parallel light beams that have been superposed one upon the other. In this description, the “perfect” plane wave refers herein to a plane wave with a mathematically perfect plane (i.e., a plane which expands to the infinity). For example, even a single-mode light beam to be emitted from a laser (such as an He—Ne laser beam) is a Gaussian beam, and its wavefront is not a mathematically perfect plane. This can be understood because a huge number of very small “perfect” plane-wave light beams are superposed one upon the other there. In this manner, even if the number of plane-wave light beams superposed in the sensing light source 19 is finite, each of those plane-wave light beams includes an infinite number of very small plane-wave light beams that have been superposed one upon the other. Consequently, the real image 18 obtained by the acousto-optic vibrometer 100 is not a set of discrete points but becomes a continuous optical image which is similar to the object 4.

As already described with reference to FIGS. 7 and 8, the light beam expansion ratio of the anamorphic prism 301 depends on the angle of incidence of a ray of light on the anamorphic prism 301 (corresponding to the angle θ1 shown in FIG. 8). That is why the diffracted light to be produced by a plurality of monochromatic rays of light that are superposed one upon the other in the plane-wave light beam is incident at different angles of incidence onto the anamorphic prism 301. As a result, the light beam expansion ratio changes from one monochromatic ray of light to another. Consequently, even if the distortion of the object image is corrected using the anamorphic prism 301, the real image 18 will still have distortion. Thus, to reduce this distortion, the acousto-optic image capture device of this embodiment includes an image processing section 20 as shown in FIG. 1. The image processing section 20 performs image processing on the image data which has been captured by the image receiving section 17, thereby correcting the residual distortion of the real image 18 and obtaining an image which is similar to the object 4.

The image processing section 20 can correct the distortion by making calculations based on the speed of sound in the medium 3 or the acousto-optic medium portion 8 and the sound-collecting and light-condensing properties of the acoustic lens system 6 and the imaging lens system 16. Also, if the object 4 is an internal body organ, for example, the medium 3 is a body tissue and the speed of sound in the medium may change significantly due to a difference between individual subjects or a difference in their body condition such as their body temperature. In that case, a reference test piece such as a medium that has been modeled based on such an individual difference or body condition difference or an elastic body, of which the shape and size are already known, is shot as the object 4 and then calibration is made so that the real image 18 thus obtained becomes an image which is exactly similar to the reference test piece. In this manner, the magnitude of the distortion correction to be made by the image processing section 20 can be determined.

However, if the F number of the acoustic lens system 6 is large (i.e., if the focal length is long for its lens aperture) or if the image capturing area on the object 4 is small, then there will be a little difference in the angle of incidence onto the anamorphic prism 301 between multiple diffracted rays of light and the light beam expansion ratio can be regarded as substantially constant. That is why in such a situation, the image processing section 20 does not have to correct the distortion of the real image 18.

Next, it will be described what the relation in size between the object 4 and the real image 18 may be in the acousto-optic vibrometer 100 of this embodiment. The acousto-optic vibrometer 100 of this embodiment may be regarded as a modified optical system of a double diffraction optical system including two optical lenses with focal lengths f and F numbers. FIG. 12A generally illustrates how a double diffraction optical system works in the field of optics.

In the double diffraction optical system shown in FIG. 12A, lenses 403 and 404 have focal lengths f and F, respectively. These two lenses 403 and 404 are arranged at two points on the optical axis 409 so as to be spaced apart from each other by a distance f+F. The optical axes of these two lenses 403 and 404 agree with the optical axis 409. Generally speaking, a convex lens with a focal length fl has focal points at two points which are located on the optical axis and each of which is located at the distance fl from the center of the lens. According to the Fourier optics, an object put at one focal point of a convex lens and an optical image at the other focal point thereof satisfy a Fourier transform relation. That is why a Fourier transformed image of the object 401 is produced by the lens 403 on a Fourier transform plane 402 which is the other focal plane (i.e., a plane which includes a focal point and which intersects at right angles with the optical axis). Since the Fourier transform plane 402 is also a focal plane of the lens 404, a Fourier transformed image of the object 401 produced on the Fourier transform plane 402 is formed on the other focal plane of the lens 404. That is to say, the optical image produced on the other focal plane of the lens 404 corresponds to what is obtained by objecting the object 401 to a dual Fourier transform. Since a dual Fourier transform is an affine mapping (i.e., a mapping technique which changes only the orientation of a figure by multiplying its size by a constant), a real image 405, which is a dual Fourier transformed image of the object 401, becomes a figure which is similar to the object 401. Also, the real image 405 appears as an inverted image of the object 401 on the focal plane of the lens 404 and the lenses 403 and 404 have mutually different focal lengths. That is why the size of the real image 405 becomes F/f times as large as that of the object 401. As can be seen, in the double diffraction optical system shown in FIG. 12A, an optical image which is similar to the object 401 appears as the real image 405, and therefore, if an image sensor such as a CCD is arranged at the focal plane of the lens 404 where the real image is produced, an image of the object 401 can be captured.

The acousto-optic vibrometer 100 of this embodiment can be regarded as a double diffraction optical system, one of the two optical systems of which has been replaced with an acoustic system. As already described with reference to FIGS. 8 and 9, this acousto-optic vibrometer 100 which produces and corrects the diffracted light 201 may be regarded as an acoustic-optical converter which converts (i.e., transfers) the amplitude distribution (acoustic pressure) on the wavefront of the plane acoustic wave 9 which is a plane wave with a wavelength λa into the amplitude distribution (light) of the distortion-compensated diffracted light 302 which is a plane wave with a wavelength λ0. That is why the optical-acoustic mixed optical system in this acousto-optic vibrometer 100 functions as an acousto-optic system in which an acoustic-optical converting section 406 for changing the wavelength from λa into λ0 is inserted between the double diffraction optical systems that are implemented as the acoustic lens system 6 and the imaging lens system 16 as shown in FIG. 12A. That is why according to the Fourier optics, even in the double diffraction acousto-optic system shown in FIG. 12B, the real image 408 also becomes an optical image which is similar to the object 407 and is produced so as to be inverted on the focal plane of the imaging lens system 16 as in FIG. 12A.

However, before and after the acoustic-optical converting section 406, the wavelength changes from λa into λ0. Thus, the size of the real image 18 becomes (F×λ0)/(f×λa) times as large as the object 4. If λ0/λa is extremely small (i.e., if the wavelength of the ultrasonic wave in the acousto-optic medium portion 8 is far longer than the wavelength of the sensing light beam 14), a decrease in the resolution of the optical image obtained by the image receiving section 17 is checked by preventing the real image from becoming too small with (F×λ0)/(f×λa) increased by setting the F/f ratio to be high.

As can be seen, this acousto-optic vibrometer transforms the scattered wave that has come from the object into a plane acoustic wave through a lens of the acoustic lens system and produces diffracted light based on a sensing light beam in which a plurality of monochromatic rays of light with mutually different traveling directions have been superposed one upon the other. By getting this diffracted light detected two-dimensionally by the image receiving section, an optical image of the object 4 can be obtained. A real image of the object can be formed passively even without performing signal processing such as the delay processing carried out by a conventional ultrasonic diagnostic apparatus. Thus, an image of the object can be obtained quickly. In addition, since a real image of the object can be formed passively, the displacement velocity distribution of the object can be measured as will be described below.

(2) Operation in Getting Displacement Velocity Distribution

Next, it will be described how the acousto-optic vibrometer 100 operates in getting the displacement velocity distribution of the object 4. By observing a so-called “Doppler shift”, i.e., a variation in frequency to be caused by an acoustic wave that has been scattered from an object in motion, the acousto-optic vibrometer 100 measures the displacement velocity distribution of the object 4. First of all, it will be described with reference to FIG. 13 how the frequency is varied by an acoustic wave that has been scattered from an object in motion while the object is being irradiated with an ultrasonic wave as the acoustic wave.

FIG. 13 schematically illustrates how the object 4 is arranged in the medium 3, an acoustic wave 2 propagates through the medium 3 and a scattered wave 5 is created. The propagation velocity of the acoustic wave 2 (i.e., the speed of sound) in the medium 3 is V. For illustrative purposes, the object 4 is supposed to be moving or being deformed either periodically or non-periodically, and an arbitrary point x on the object 4 is supposed to be being displaced at a velocity v(x) (where the point x and the velocity v(x) are vector quantities) at a certain point in time. The velocity v(x) may have a different magnitude and direction from one point x to another.

The acoustic wave 2 is a plane acoustic wave with a frequency f. Suppose an observer 231 who is standing still with respect to the medium 3 found that the frequency of the scattered wave 5 to be created by getting the acoustic wave 2 radiated reflected or diffracted from the surface or inside of the object 4 was f″. The traveling direction of the acoustic wave 2 is supposed to be eᵢ (where eᵢ is a vector, of which the magnitude is one) and a direction vector indicating the direction from the point x toward the observer 231 is supposed to be eₒ (where eₒ is also a vector, of which the magnitude is one).

If the form of the object 4 changes with time, the respective frequencies f and f″ of the acoustic wave 2 and the scattered wave 5 generally have mutually different values. This variation in frequency Δf(x)=f″−f is called a “Doppler shift” 233. The Doppler shift 233 is a function of the three vectors eᵢ, eₒ and v(x) described above, and its specific function form is given by the following Equations (4):

$$Δ f ( x ) = f - {f}^{″} \\ {f}^{″} = \frac{V - v ( x ) \cdot {e}_{i}}{V - v ( x ) \cdot {e}_{0}} f \qquad ( 4 )$$

In Equations (4), v(x)·eᵢ represents the inner product of the two vector quantities v(x) and eᵢ, and v(x)·eₒ represents the inner product of the two vector quantities v(x) and eₒ.

If the velocity vectors v(x) at respective points x on the object 4 are different from each other, the scattered waves 5 that have come from those points have mutually frequencies. That is why if the Doppler shift 233 of a scattered wave that has come from a point x is measured, the velocity vector v(x) at the point x (more exactly, the projection component of v(x) onto the directions of the vectors eᵢ and eₒ) can be estimated conversely. As a result, the distribution of displacement velocities can be measured.

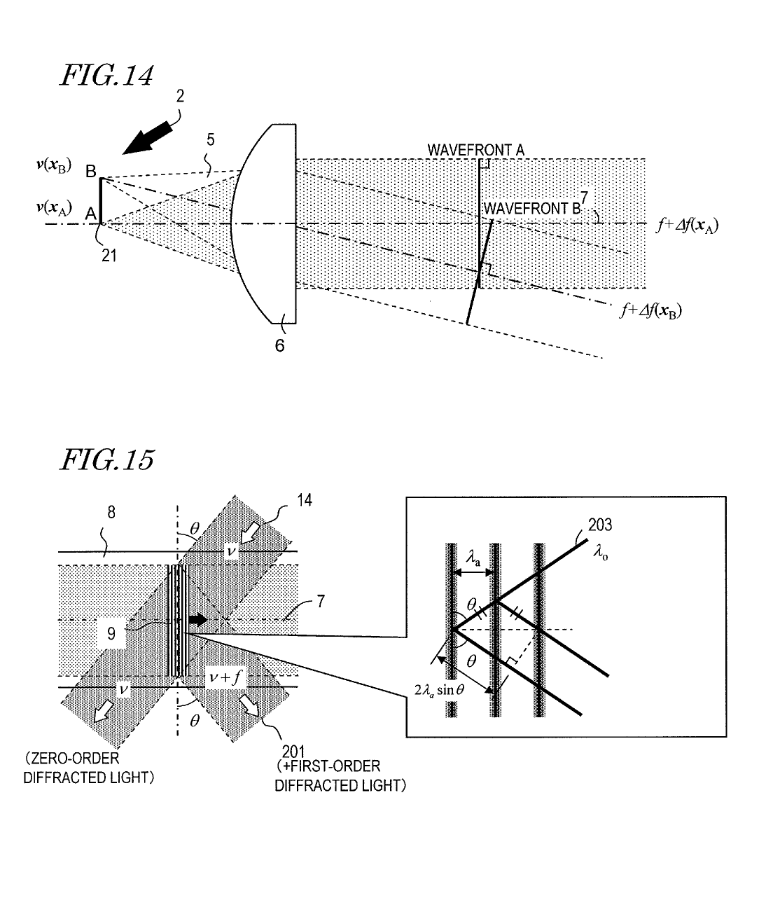

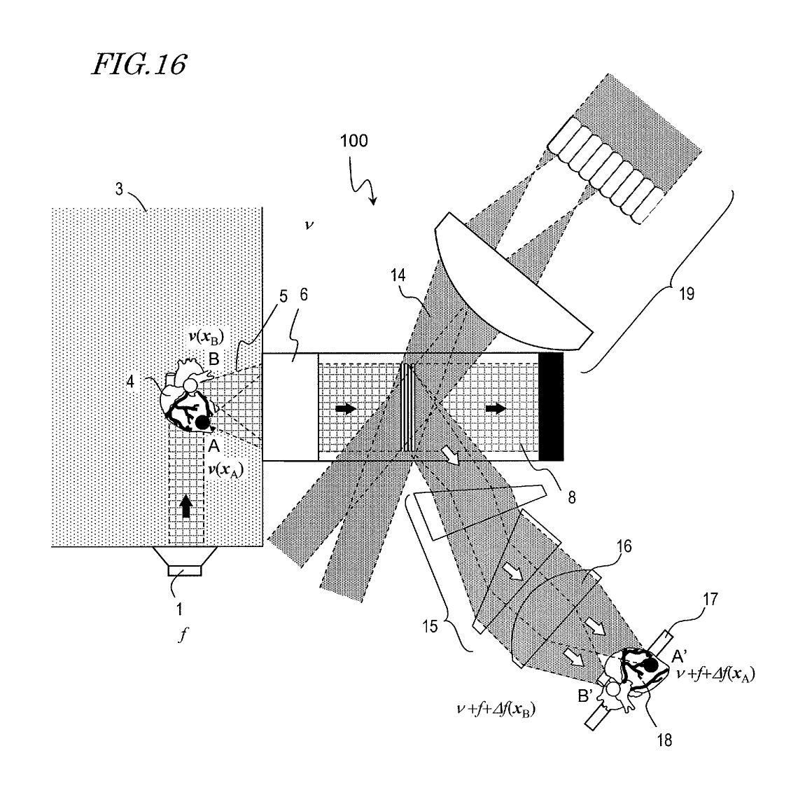

The acousto-optic vibrometer 100 detects the Doppler shift 233 which has been caused by the displacement of the object 4 as a flickering period of the light intensity of the real image 18. The Doppler shift 233 may be detected by the image processing section 20 based on an electrical signal supplied from the image receiving section 17. For illustrative purposes, two different points A and B on the object 4 are supposed to be moving at mutually different velocity vectors v(x_{A}) and v(x_{B}) as shown in FIG. 14. If the object 4 is irradiated with an acoustic wave 2 at a frequency f, scattered waves 5 are created from the two points A and B. If those two points A and B are located on the focal plane 21 of the acoustic lens system 6, those scattered waves 5 coming from those two points are transformed into plane acoustic waves with mutually different wavefronts A and B. As these two points A and B have mutually different velocity vectors v(x_{A}) and v(x_{B}), the scattered waves 5 coming from the respective points are subjected to respectively different Doppler shifts Δf(x_{A}) and Δf(x_{B}) as already described with reference to FIG. 13. As a result, the plane acoustic waves with wavefronts A and B that have been transformed by the acoustic lens system 6 have mutually different frequencies f+Δf(x_{A}) and f+Δf(x_{B}). It should be noted that since a scattered wave 5 is created from every point x on the focal plane 21, the scattered wave 5 will be a plane acoustic wave 9 in which plane acoustic waves having the frequency f+Δf(x) and having mutually different traveling directions are superposed one upon the other when the scattered wave 5 has passed through the acoustic lens system 6.

As already described with reference to FIG. 11, by irradiating each of the plane acoustic waves with the wavefronts A and B with the sensing light beam 14 at such an angle that satisfies the Bragg diffraction condition, diffracted light 201 is produced from each of those plane waves. The diffracted light 201 has information about the acoustic pressure intensity distribution and propagation direction of each plane acoustic wave and also has information about the Doppler shift Δf(x) of each plane acoustic wave. As shown in FIG. 15, if a sensing light beam 14 having a frequency ν is incident on the plane acoustic wave 9 having a frequency f at such an angle θ that satisfies the Bragg diffraction condition, diffracted light 201 is produced. Since the plane acoustic wave 9 is a traveling acoustic wave, this diffraction phenomenon is the same physical phenomenon as the diffraction phenomenon of an acousto-optic modulator. That is to say, the frequency of the diffracted light 201 changes from that of the sensing light beam 14 by the frequency f of the plane acoustic wave 9 (i.e., undergoes a frequency modulation). Specifically, if the diffracted light 201 is +first-order diffracted light, the frequency of the diffracted light 201 becomes ν+f, which has increased by the frequency f of the plane acoustic wave 9. It should be noted that the frequency of zero-order diffracted light does not change (but remains v). The frequency of −first-order diffracted light decreases by the frequency f of the plane acoustic wave 9 and becomes v−f. As can be seen, the diffracted light 201 has not only information about the acoustic pressure intensity distribution and propagation direction of the plane acoustic wave 9 but also frequency information as well.

Consequently, the luminous points that have converged at respective points on the real image 18 generated by the acousto-optic vibrometer 100 have frequencies that change with the displacement velocity of the object 4. To describe this, a ray of light tracking diagram, from which the reference light source 23 is removed, is shown in FIG. 16. As described above, the acousto-optic vibrometer 100 projects a real image 18 which is similar to the object 4 onto the photosensitive plane of the image receiving section 17. Now look at two points A and B on the object 4. Since these two points A and B are being displaced with mutually different velocity vectors v(x_{A}) and v(x_{B}), the scattered waves 5 that have come from those points A and B are subjected to different Doppler shifts Δf(x_{A}) and Δf(x_{B}) and are transformed by the acoustic lens system 6 into plane acoustic waves with frequencies f+Δf(x_{A}) and f+Δf(x_{B}), respectively. These plane acoustic waves are illuminated with a sensing light beam 14 with a frequency ν, thus producing diffracted light beams associated with the points A and B through the Bragg diffraction. Each of these diffracted light beams has gone through a frequency modulation using a plane acoustic wave. As the +first-order diffracted light beam is used to produce the real image 18 according to this embodiment, the frequencies of the respective diffracted light beams become v+f+Δf(x_{A}) and ν+f+Δf(x_{B}), respectively. These diffracted light beams are converged by the distortion compensating section 15 and the imaging lens system 16 onto points A′ and B′ on the real image 18 as luminous points. These luminous points have mutually different frequencies v+f+Δf(x_{A}) and ν+f+Δf(x_{B}). Consequently, the luminous points corresponding to the displacement velocity vector distribution v(x) on the object 4 and forming the real image 18 have a frequency distribution ν+f+Δf(x).

That is why by measuring the frequency distribution ν+f+Δf(x), the velocity vector distribution v(x) on the object 4 can be measured. However, since ν+f+Δf(x) is a very high frequency which is almost as high as the frequency of visible radiation, it is generally not easy to measure that high frequency. For that reason, the acousto-optic vibrometer 100 superposes the reference light beam 24 that has been produced by the reference light source 23 on the distortion-compensated diffracted light 302 and uses interference to measure Δf(x). For example, if two monochromatic rays of light with frequencies ν and ν+Δν (where v>>Δv) are superposed one upon the other to interfere with each other, a beat light beam, of which the light intensity varies at a differential frequency Δv, is produced. The acousto-optic vibrometer 100 uses this principle.

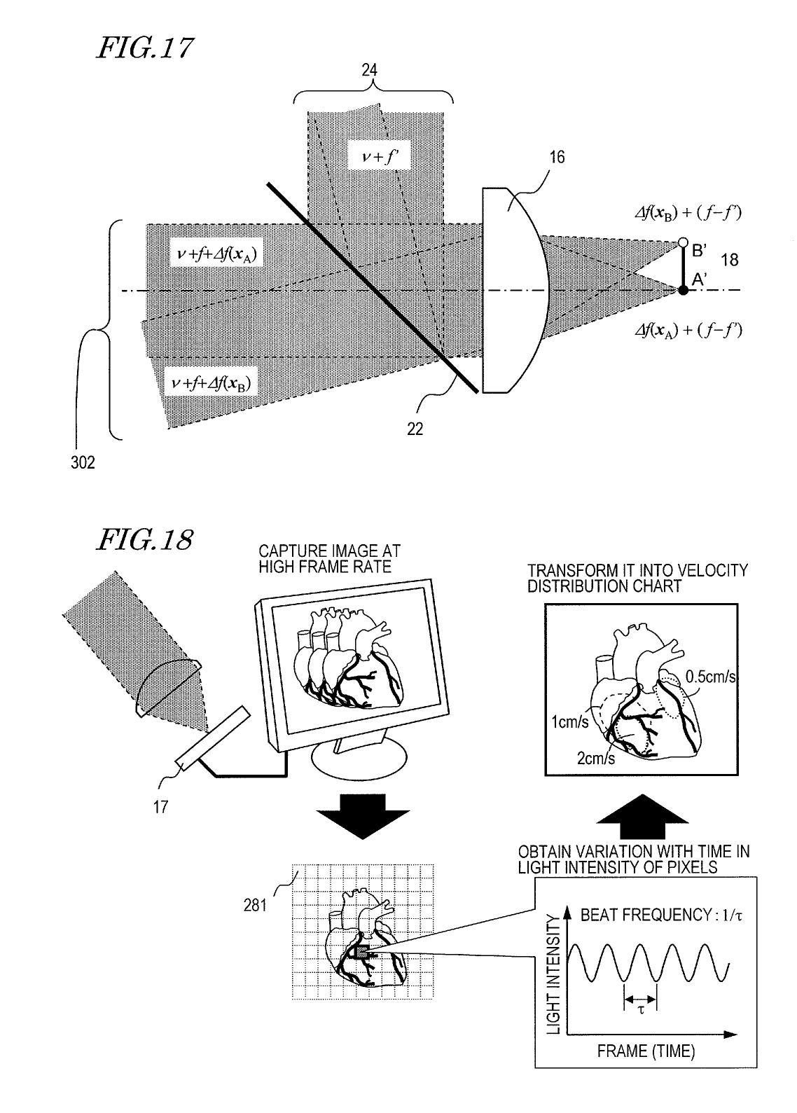

FIG. 17 schematically illustrates how the reference light beam 24 that has been produced by the reference light source 23 is superposed by the beam splitter 22 on the distortion-compensated diffracted light beams 302 and the beat light beam thus generated is converged by the imaging lens system 16 to form a real image 18. As in FIG. 16, the two points on the real image 18 corresponding to the two points A and B on the object 4 are A′ and B′, respectively, in FIG. 17, too, and the distortion-compensated diffracted light beams 302 that form luminous points on the two points A′ and B′ are subjected to the frequency modulation and have frequencies v+f+Δf(x_{A}) and ν+f+Δf(x_{B}), respectively. As already described with reference to FIG. 6, the reference light beam 24 is a light beam in which plane-wave light beams with various traveling directions and having the frequency ν+f′ are superposed one upon the other. That is why by getting the reference light beam 24 superposed on the distortion-compensated diffracted light beams 302 by the beam splitter 22, plane-wave light beams, which have the frequency ν+f′ and of which the wavefronts agree with those of the plane-wave light beams that form the luminous points A′ and B′, can be made to interfere with each other and a beat light beam can be generated. As a result, the luminous points A′ and B′ on the real image 18 form a beat light beam, of which the intensity varies with the differential frequency between the distortion-compensated diffracted light beams 302 and the reference light beam 24. More specifically, the frequencies of variation in the light intensity of the luminous points A′ and B′ become Δf(x_{A})+(f−f′) and Δf(x_{B})+(f−f′), respectively. One period of these light intensity variations is long enough to get the measurement done as intended. As can be seen, the real image 18 produced by the acousto-optic vibrometer 100 becomes an optical image comprised of luminous points, of which the flickering velocity changes from one point to another. More specifically, the real image 18 becomes an optical image which flickers at the beat frequency Δf(x)+(f−f′) corresponding to the velocity vector distribution v(x) on the object 4.

Next, it will be described with reference to FIG. 18 how the acousto-optic vibrometer 100 measures the beat frequency distribution Δf(x)+(f−f′) of the real image 18. As the image receiving section 17 of the acousto-optic vibrometer 100, a high-speed image sensor, of which the frame rate is at least a few times as high as the beat frequency Δf(x)+(f−f′), is used. As will be described below about an exemplary specific configuration, a high-speed image sensor including a global shutter such as a CCD (solid-state image sensor) in which pixels are arranged two-dimensionally may be used. In this description, the global shutter refers herein to an image getting method in which every pixel is captured at the same timing.

First of all, images are captured sequentially by the image receiving section 17 as at least two consecutive frames. Next, based on the image data thus obtained (consisting of multiple frames), a variation in light intensity with time is measured on a pixel (281) by pixel basis. Then, based on the variation in light intensity with time thus measured, the beat frequency is calculated on a pixel by pixel basis.

Since the frequency f of the acoustic wave 2 and the modulated frequency f′ of the monochromatic light emitted from the acousto-optic modulator 214 are known in advance, the Doppler shift Δf(x) can be calculated based on the pixel-by-pixel beat frequency. And based on the Doppler shift Δf(x) thus obtained, the velocity vector distribution v(x) on the object 4 is calculated. Specifically, by the following Equations (5) to be derived from Equations (4), the velocity vector distribution v(x) is calculated:

$$v ( x ) \cdot \{ {e}_{i} + ( k - 1 ) {e}_{0} \} = kV \\ k = \frac{Δ f ( x )}{f} \qquad ( 5 )$$

The constant k in Equations (5) can be obtained based on the Doppler shift Δf(x) thus measured and the frequency f of the acoustic wave 2. The vectors eᵢ and eₒ can be obtained based on the configuration of this acousto-optic vibrometer 100 and the images captured. That is why based on the Doppler shift Δf(x) measured, the magnitude of the velocity vector distribution v(x) in the direction of the vector eᵢ+(k−1)eₒ can be obtained. In this manner, the acousto-optic vibrometer 100 can measure the magnitude of the velocity vector distribution v(x) in the direction of the vector eᵢ+(k−1)eₒ based on the beat frequency distribution Δf(x)+(f−f′) of the real image 18.

If not the magnitude of the velocity vector distribution v(x) of the object 4 in the direction of the vector eᵢ+(k−1)eₒ but the vector quantity of the velocity vector distribution v(x) of the object 4 needs to be measured, then the following configuration and calculating method are used. As can be seen from Equations (5), if the beat frequency distribution Δ^{m}f(x) is measured using m₀ different eᵢ=eᵢ^{m} (where m=1, 2, . . . or m₀ and m₀ is an integer that is equal to or greater than three) and if m₀ equations obtained by substituting m₀ k=k^{m}=Δ^{m}f(x)/f (where m=1, 2, . . . or m₀ and m₀ is an integer that is equal to or greater than three) measuring data and eᵢ=eᵢ^{m}, eₒ into Equations (5) are satisfied simultaneously, a velocity vector distribution v(x) can be obtained as a three-component vector quantity. The reason is that Equations (5) are linear equations with respect to a three-component unknown function v(x) and therefore always have a solution with respect to each of m₀≧3 independent equations. In this manner, the velocity vector distribution v(x) as a vector quantity can be measured. It should be noted that the three or more different eᵢ (=eᵢ^{m}) are obtained by providing m₀ (=3) different acoustic wave sources 1 as shown in FIG. 19, for example. In that case, to distinguish the acoustic waves 2 emitted from those different acoustic wave sources 1 from each other, the acoustic waves 2 emitted from those different acoustic wave sources 1 suitably have mutually different frequencies. Or if the different acoustic wave sources 1 emit acoustic waves 2 with the same frequency, the timings of emission may be shifted from each other.

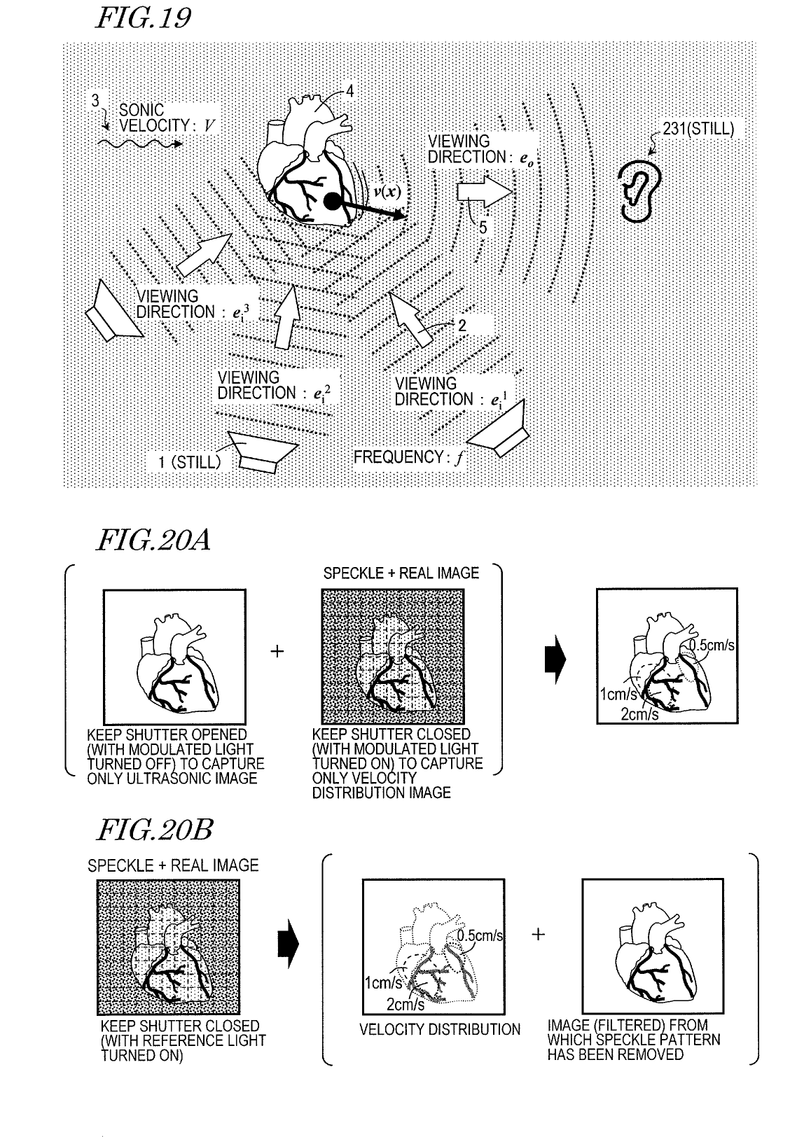

The acousto-optic vibrometer 100 may capture images based on the acoustic waves and may measure the velocity vector distribution v(x) in the following manner, for example. As shown in FIG. 20A, by using two image data that were captured by the image receiving section 17 at mutually different points in time, an image of the object is captured and the velocity vector v(x) is measured based on each of the image data. The points in time at which the image data are obtained are suitably so close to each other that the object 4 can be regarded as standing still during the measurement. More specifically, first of all, the shutter 215 (see FIG. 6) of the reference light source 23 is closed and the object 4 is shot using an acoustic wave. Next, the shutter 215 is opened to obtain the image data on which the reference light beam 24 has been superposed, and the velocity vector distribution v(x) is measured by the method that has already been described with reference to FIG. 18. As described above, the light intensity distribution of the reference light beam 24 emitted from the reference light source 23 is a uniform one, and therefore, includes a speckle pattern. That is why the real image 18 to be generated when the reference light beam 24 is superposed and makes interference is inferior to the real image 18 on which such a speckle pattern is superposed on the real image of the object 4 and no reference light beam 24 is superposed. As can be seen, by shooting the object and measuring the velocity vector v(x) in two different frames, a high-definition image and a displacement velocity distribution image can be obtained.

Alternatively, as shown in FIG. 20B, the shutter 215 of the reference light source 23 may always be kept open and only an optical image on which a speckle pattern caused by the reference light beam 24 is superposed may be obtained. In that case, the velocity vector distribution v(x) is measured based on the image data thus obtained by the method that has already been described with reference to FIG. 18 and the speckle pattern is removed by image filtering, thereby reproducing an optical image of the object 4 on which no reference light beam 24 is superposed. A technique for removing a speckle pattern by image processing has already been established. For example, by using a speckle noise reduction filter, a speckle-pattern-free optical image, i.e., an optical image of the object 4 on which no reference light beam 24 is superposed, can be reproduced with high precision. According to this method, an image is captured and the displacement velocity distribution v(x) is measured based on a single piece of image data, and therefore, the measurement can get done much more quickly.

As can be seen from the foregoing description, the acousto-optic vibrometer of this embodiment superposes a reference light beam in which a plurality of monochromatic rays of light with mutually different traveling directions are superposed one upon the other on the diffracted light beam described above, and detects the superposed light two-dimensionally at the image receiving section. As a result, the Doppler shift caused by the displacement of any part of the object can be detected as a variation in luminance in that part.

3. Exemplary Specific Configuration

Hereinafter, an exemplary specific configuration of an acousto-optic vibrometer 100 according to this embodiment will be described.

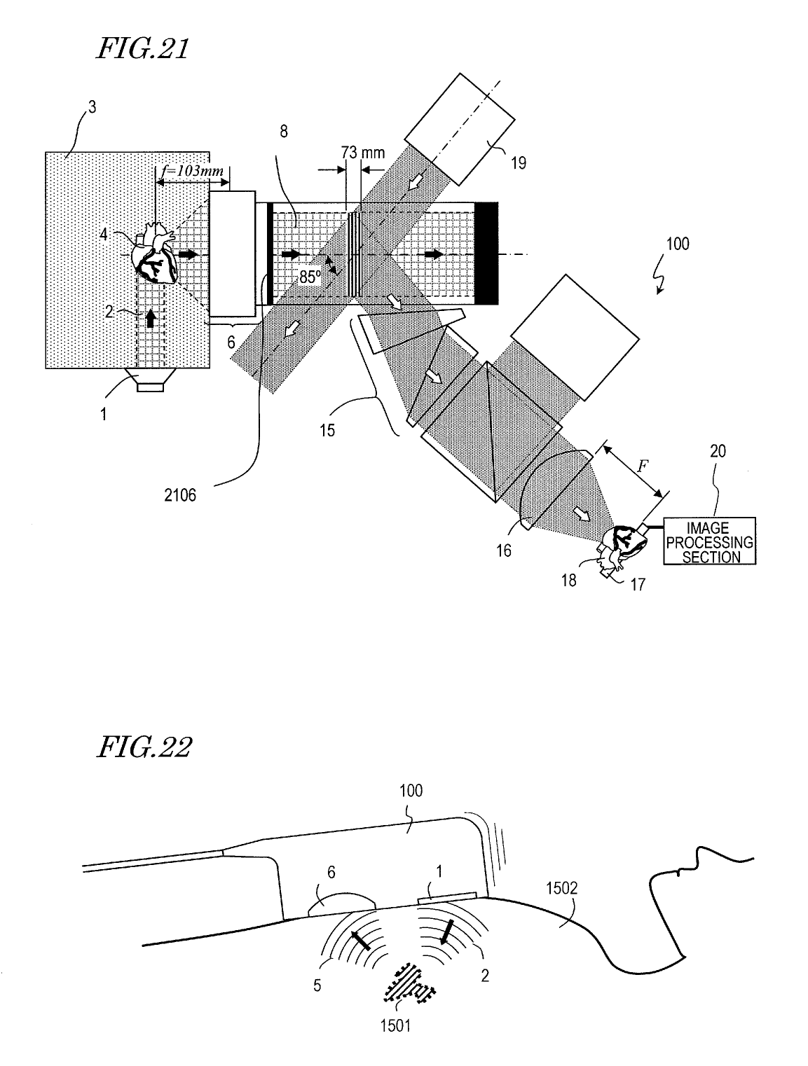

FIG. 21 illustrates an exemplary specific configuration for the acousto-optic vibrometer 100. The configuration of the apparatus shown in FIG. 21 can be used effectively to irradiate an internal body organ with acoustic wave 2 from outside of a person's body and capture an image of the organ such as a cardiac wall or an arterial wall or monitor its movement and displacement as in an ultrasonic diagnostic apparatus.

In monitoring an internal body organ as shown in FIG. 21, the medium 3 is a body tissue. The acoustic wave source 1 emits a burst signal which may be twenty sine waves with a frequency of 13.8 MHz, for example, as the acoustic wave 2. This burst signal may have a signal duration of 1.4 μsec (1.4×10⁻⁶ seconds), for example. Also, since the speed of sound in the body tissue is approximately 1500 m/s, the acoustic wave 2 in the body tissue has a wavelength of approximately 110 μm and the physical signal length as measured parallel to the traveling direction of the acoustic wave 2 is approximately 2.2 mm. Consequently, in the acousto-optic vibrometer shown in FIG. 21, the object 4 which is vibrating at a frequency of several hundred kHz at maximum can be shot at a spatial resolution of several hundred μm.

As the acousto-optic medium portion 8, a nanoporous silica with a speed of sound of 50 m/s is used. The nanoporous silica has so low a speed of sound and so short an ultrasonic wave propagation wavelength that a large angle of diffraction can be obtained. In addition, the nanoporous silica is sufficiently transparent with respect to an He—Ne laser beam with a wavelength of 633 nm, and can be used effectively in the exemplary configuration shown in FIG. 21. Alternatively, a fluorine-based solvent such as fluorinert that can transmit light well also has as low a speed of sound (of approximately 630 m/s) and may also be used no less effectively as the acousto-optic medium portion 8.

As described above, an He—Ne laser with a wavelength of 633 nm may be used as the monochromatic light source 11. In that case, the angle of diffraction of the first-order diffracted light becomes 5 degrees in the exemplary configuration shown in FIG. 21. If the angle of diffraction of the first-order diffracted light is 5 degrees, then the beam expansion ratio to be achieved by the image distortion compensating section 15 is approximately 5.74, which is a value that can be compensated for by a retailed anamorphic prism.

If an upper limit is put legislatively on the acoustic pressure of an acoustic wave that can be radiated inside the body, an image sensor with high sensitivity is suitably used as the image receiving section 17 to observe a tissue in which the diffracted light produced has low light intensity. Also, in view of image quality and light quantity considerations, in order to capture a real image 18 at the moment when the plane acoustic wave 9 passes through the sensing light beam 14 and to monitor the motion of the object 4 with the magnitude of Doppler shift measured by sequential shooting, an image sensor that can shoot at high speeds is used as the image receiving section 17. The frame rate of the image sensor to be used as the image receiving section 17 may be selected appropriately according to the velocity of displacement of the object 4. For example, to detect the motion of the heart (with a maximum displacement velocity of approximately 0.07 m/s), a high-speed CCD (charge-coupled device) image sensor with a frame rate of approximately 2000 frames per second may be used as the image receiving section 17. If the luminance of the real image 18 is too low to capture a good image easily, an image intensifier may be arranged just before the image sensor to increase the luminance of the real image 18. Alternatively, a monochromatic light source 11 with an even higher output may be used. To minimize the deformation of the real image 18 due to the displacement of the object 4, the image sensor suitably has a global shutter as described above.

As already described, an ultrasonic wave will be reflected from an interface between two acoustic media with mutually different acoustic impedances to cause a decrease in the intensity or quality of the real image 18. The greater the difference in acoustic impedance at the interface, the more significantly the acoustic wave will be reflected. That is why in the exemplary configuration shown in FIG. 21, a matching layer B 2106 functioning as an antireflection film is provided at the interface between the acoustic lens system 6 and the medium 3. For example, the body tissue as the medium 3 has approximately the same acoustic properties (including a speed of sound of 1500 m/s and a density of 1 g/cm³) as water. If a nanoporous silica with a speed of sound of 50 m/s and a density of 0.11 g/cm³ is used as the acousto-optic medium portion 8, a thin film having a thickness of 6.7 μm and made of a nanoporous silica with a speed of sound of 367 m/s and a density of 0.27 g/cm³ is stacked so as to contact with a parallel plate acousto-optic medium portion 8 made of polystyrene with a thickness of 140 μm (i.e., a one wavelength matching film), thereby obtaining the matching layer B 2106.

As already described above, the real image 18 is (F×λ₀)/(f×λₐ) times as large as the object 4, the wavelength of the light λ₀ is 633 nm, and an ultrasonic wave of 13.8 MHz in the nanoporous silica with a speed of sound of 50 m/s has a wavelength λₐ of 3.6 μm. That is why if a real image 18, of which the size is one-fifth of the object 4, needs to be obtained on the image receiving section 17, then F/f=1.14, because (F×633)/(f×3600)=1.5 is satisfied. That is why if an acoustic lens system 6 with a focal length of 103 mm is used, then an imaging lens system 16, of which the focal length is 117 mm (=1.14×103), may be used.

As already described with reference to FIG. 10, if the ratio of magnification (F×λ₀)/(f×λₐ) of the real image 18 to the object 4 is increased, the focal length of the imaging lens system 16 may increase and the acousto-optic vibrometer 100 may have a bigger overall size. In that case, by using a reflective optical system such as a Cassegrain optical system as the imaging lens system 16, the overall size of the imaging lens system 16 can be reduced. In addition, the imaging lens system 16 and the real image 408 can be arranged so that their distance is shorter than the actual focal length F. As a result, the size of the acousto-optic vibrometer 102 can be reduced.

Optionally, the size of the acousto-optic vibrometer 100 can also be reduced by arranging the acoustic lens system 6 and the imaging lens system 16 so that their distance is shorter than f+F. As already described with reference to FIG. 12B, the optical-acoustic mixed optical system of the acousto-optic vibrometer 100 can be regarded as a double diffraction optical system in the field of optics. According to the basic arrangement of a double diffraction optical system, the acoustic lens system 6 and the imaging lens system 16 are arranged so as to be spaced apart from each other by the sum (f+F) of the focal lengths of the respective lenses. However, even if the distance between the acoustic lens system 6 and the imaging lens system 16 is set to be a different value from f+F, an optical image formed by the real image 18 is not affected. That is to say, as long as the optical image of the real image 18 is obtained as a light intensity distribution (or unless the phase distribution information of the real image 18 is observed), the distance between the acoustic lens system 6 and the imaging lens system 16 may be shorter than f+F, and the size of the acousto-optic vibrometer 100 can be further reduced.

An exemplary specific application of this acousto-optic vibrometer 100 will be described with reference to FIG. 22. As shown in FIG. 22, this acousto-optic vibrometer 100 may be used as a visualizing apparatus to monitor an internal body organ 1501 non-invasively in making a physical or medical checkup, for example. In the example illustrated in FIG. 22, as in a conventional ultrasonic probe, the acousto-optic vibrometer 100 is integrated in a single unit. In that unit, either everything or everything but the monochromatic light source 11 of the apparatus shown in FIG. 1 is integrated together. In capturing an image, this acousto-optic vibrometer 100 is brought into contact with the body surface of a person under test 1502 so that an acoustic wave 2 which has been created from the acoustic wave source 1 outside of his or her body is transmitted inside his or her body. In this case, attenuation of the acoustic wave 2 by being reflected from the body surface may be reduced in order to increase the sensitivity. This can be done by matching the acoustic impedance of the internal body tissue and the material of the contact face of the acoustic wave source 1 to each other at the interface between the contact face of the acousto-optic vibrometer 100 and the body surface. Such matching can get done by adopting a matching gel or cream which is used in a conventional ultrasonic diagnostic apparatus or by providing an acoustic impedance matching layer on the surface of the acoustic wave source 1.

Part of the acoustic wave 2 propagating through his or her body tissue gets scattered from an organ 1501 to turn into a scattered wave 5. The scattered wave 5 then reaches the acoustic lens system 6 and is transformed into a plane wave by the acoustic lens system 6. As a result of the operation of the acousto-optic vibrometer 100 described above, an image of the organ 1501 is obtained. An image of the organ 1501 which is located within a plane that intersects at right angles with the acoustic axis 7 (not shown) of the acousto-optic vibrometer 100 and outside of the image capturing region can be captured by moving the acousto-optic vibrometer 100 on the body surface just like a conventional ultrasonic probe. Also, another organ located at a different depth in the person's body can also be shot by the focal length adjusting mechanism 2108 of the acoustic lens system 6.

The acousto-optic vibrometer 100 of this embodiment may be modified in various manners. For example, the acousto-optic vibrometer 100 may include a focal length adjusting mechanism which is provided for the imaging lens system. 16. Then, the acousto-optic vibrometer 100 can perform the zoom function. Specifically, the ratio of magnification of the real image 18 to the object 4 can be changed. As a result, a more detailed region or a broader region of the object 4 can be observed easily.

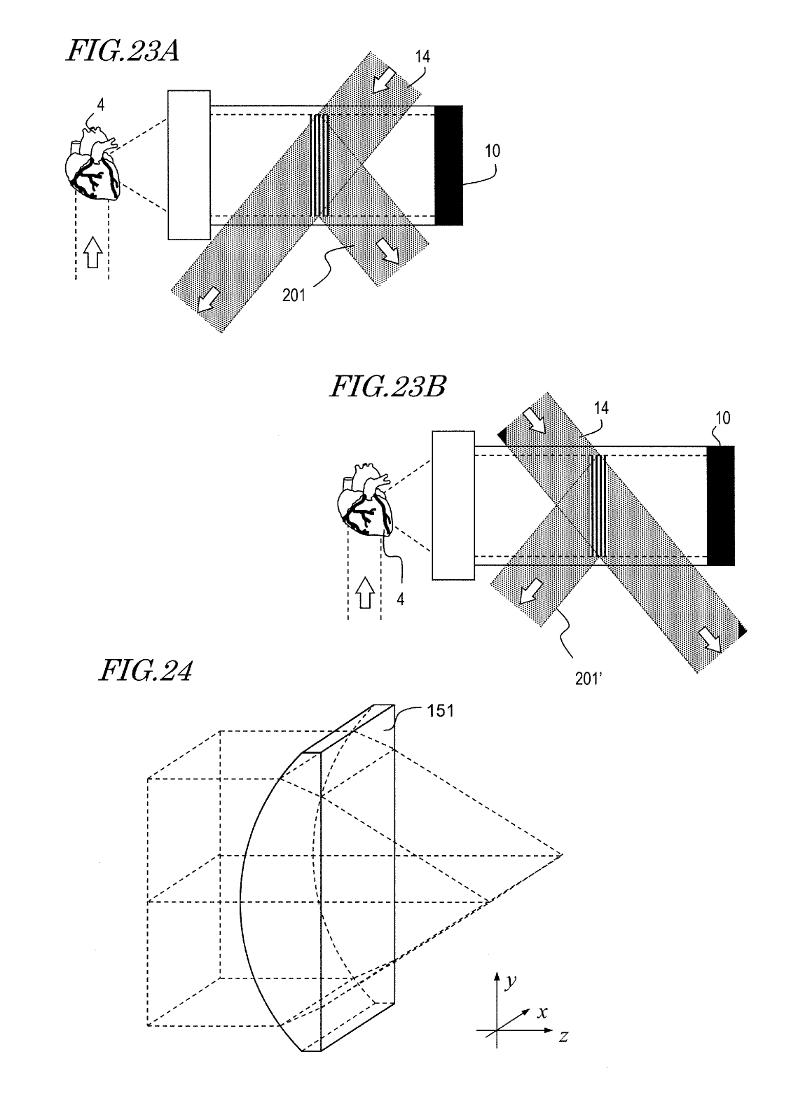

Also, in the embodiment described above, a sensing light beam 14 is radiated from the acoustic wave absorbing end 10 toward the object 4 as shown in FIG. 23A. Alternatively, a sensing light beam 14 may also be radiated from the object 4 toward the acoustic wave absorbing end 10 as shown in FIG. 23B. In that case, diffracted light 201′ goes out toward the object 4. Also, the real image obtained will be a mirror image of the real image to be produced with the configuration shown in FIG. 23A. In that case, the former real image will be symmetrical to the latter with respect to the paper on which FIG. 12B is drawn. That is why to obtain a real image 18 of the object 4 in a proper orientation, the image shot needs to be either reflected once by a plane mirror, or processed by the image processing section 20, to produce an optically inverted mirror image.

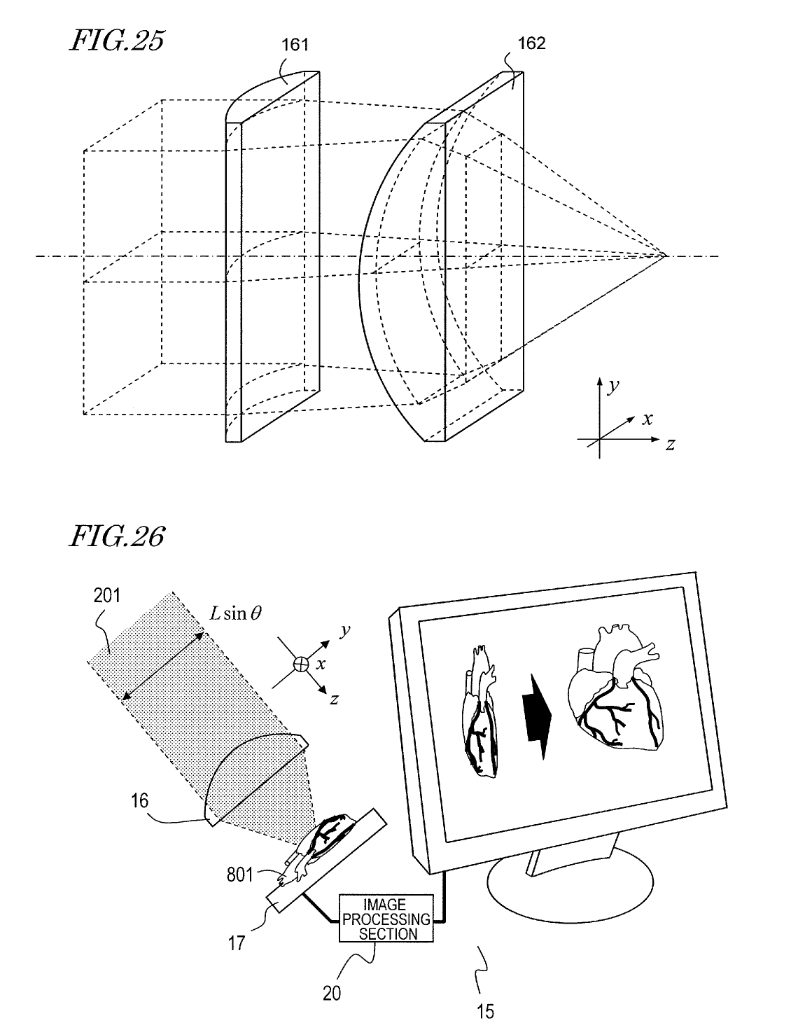

Furthermore, in the embodiment described above, an anamorphic prism 301 is used as the image distortion compensating section 15. However, any other optical system with a similar function may also be used instead as the image distortion compensating section 15. For example, the image distortion compensating section 15 may also be formed by two condensing cylindrical lenses. As shown in FIG. 24, a cylindrical lens 151 is an optical element which functions as a condenser lens within a plane that is parallel to the yz plane of the coordinate system defined on the drawing but which does not have a condensing function within a plane that is parallel to the xz plane. As shown in FIG. 25, an optical system as a combination of two cylindrical lenses, of which the planes with a light condensing function intersect with each other at right angles, functions as both the image distortion compensating section 15 and as the imaging lens system 16. The coordinate system shown in FIG. 25 agrees with the coordinate system shown in FIG. 9B. The cylindrical lenses 161 and 162 are arranged in the orientations shown in FIG. 25 with respect to this coordinate system. The cylindrical lens 161 has a longer focal length than the cylindrical lens 162, and these two lenses 161 and 162 have the same focal point. The optical system formed by these cylindrical lenses 161 and 162 functions as an optical system which images the light at different ratios on yz and xz planes. By selecting the focal lengths of these two lenses to compensate for the oblateness sin θ of the light beam shown in FIG. 3 and to make the ratio of the image in the y-axis direction to the image in the x-axis direction l/sin θ, a real image 18 which is similar to the object 4 can be produced just like the anamorphic prism 301. Specifically, the focal lengths of the two lenses 161 and 162 may be set so that the focal length of the cylindrical lens 162 becomes sin θ times as long as that of the cylindrical lens 161. In that case, the focal length of the cylindrical lens 161 is determined by the ratio of magnification of the real image 18 to the object 4.

It should be noted that if the image distortion compensating section 15 and the imaging lens system 16 are replaced with the optical system shown in FIG. 25, no distortion correction needs to be made by the image processing section 20 as long as the distortion is corrected sufficiently by the cylindrical lenses 161 and 162.

As can be seen from the foregoing description, the acousto-optic vibrometer 100 can not only obtain an image with high definition quickly but also monitor the elastic property of an organ without requiring a special testing environment that depends on the object under test. In addition, the acousto-optic vibrometer 100 can also obtain the distribution of displacement velocities of respective parts of the organ.

### Embodiment 2

Hereinafter, a second embodiment of an acousto-optic vibrometer according to the present invention will be described. The acousto-optic vibrometer of this second embodiment is the same as the acousto-optic vibrometer of the first embodiment except that the image distortion compensating section 15 has a different configuration. Thus, the following description of the second embodiment will be focused on only the configuration of the image distortion compensating section 15. FIG. 26 schematically illustrates a configuration for the image distortion compensating section 15 of this embodiment.

As already described for the first embodiment, in the acousto-optic vibrometer 100, a large number of plane-wave light beams with mutually different traveling directions are superposed one upon the other in the sensing light beam 14 produced by the sensing light source 19. In the following description, special attention will be paid to some of those plane-wave light beams that are parallel to the optical axis 13.

As described with reference to FIG. 9A, supposing the angle of diffraction is θ, the diffracted light 201 produced by Bragg diffraction has had its size reduced sin θtimes in the y-axis direction in the coordinate system that has been set in FIG. 9A. That is why if the diffracted light 201 were imaged as it is by the imaging lens system 16, then the real image 18 would be distorted in the y-axis direction and no real image 18 that is similar to the object 4 could be obtained. For that reason, the image distortion compensating section 15 compensates for the distortion of the light beam by multiplying the size by 1/sin θ in the y-axis direction in the coordinate system that has been set in FIG. 9A. In the first embodiment described above, the image distortion compensating section 15 is implemented as an optical system including an anamorphic prism or a cylindrical lens which is an optical element.

According to this embodiment, however, the function of the image distortion compensating section 15 is performed by a non-optical method. Specifically, as shown in FIG. 26, the distorted diffracted light 201 is imaged as it is by the imaging lens system 16. In this case, the real image 801 is distorted in the y-axis direction but a real image 801 in that state is gotten as it is by the image receiving section 17. The image processing section 20 receives an electrical signal representing the real image 801 from the image receiving section 17 and removes the image distortion from the real image 801 through image processing. For example, by performing image processing to multiply the real image 801 by 1/sin θ in the y direction on the coordinate system shown in FIG. 26, an image which is similar to the object 4 is generated.

According to this embodiment, the number of optical elements to use can be reduced. In addition, there is no need to leave any space in the image processing section 20 for the optical image distortion compensating section 15 to perform the function of the image distortion compensating section 15. As a result, an acousto-optic vibrometer of a reduced size can be provided at a lower manufacturing cost.

It should be noted that if the angle of diffraction θ is small, the object 4 is shot so that its image will be significantly expanded in the y-axis direction in the coordinate system defined in FIG. 9A on the image capturing plane of the image receiving section 17. That is why after having been subjected to the image processing, the resolution of the image in the x-axis direction will be different from its resolution in the y-axis direction. For that reason, both the optical image distortion compensating section 15 shown in FIG. 9B and the image distortion compensating section 15 that gets image processing performed by the image processing section 20 may be used in combination.

Furthermore, if the anamorphic prism 301 is used as the optical image distortion compensating section 15 shown in FIG. 9B and if the image distortion compensating section 15 of this embodiment that performs image processing is also used, then a field of curvature will be produced because the angles of incidence of a lot of diffracted rays of light 201 on the anamorphic prism 301 are different from each other. That is why the aberration may also be corrected by the image processing section 20.

### Embodiment 3

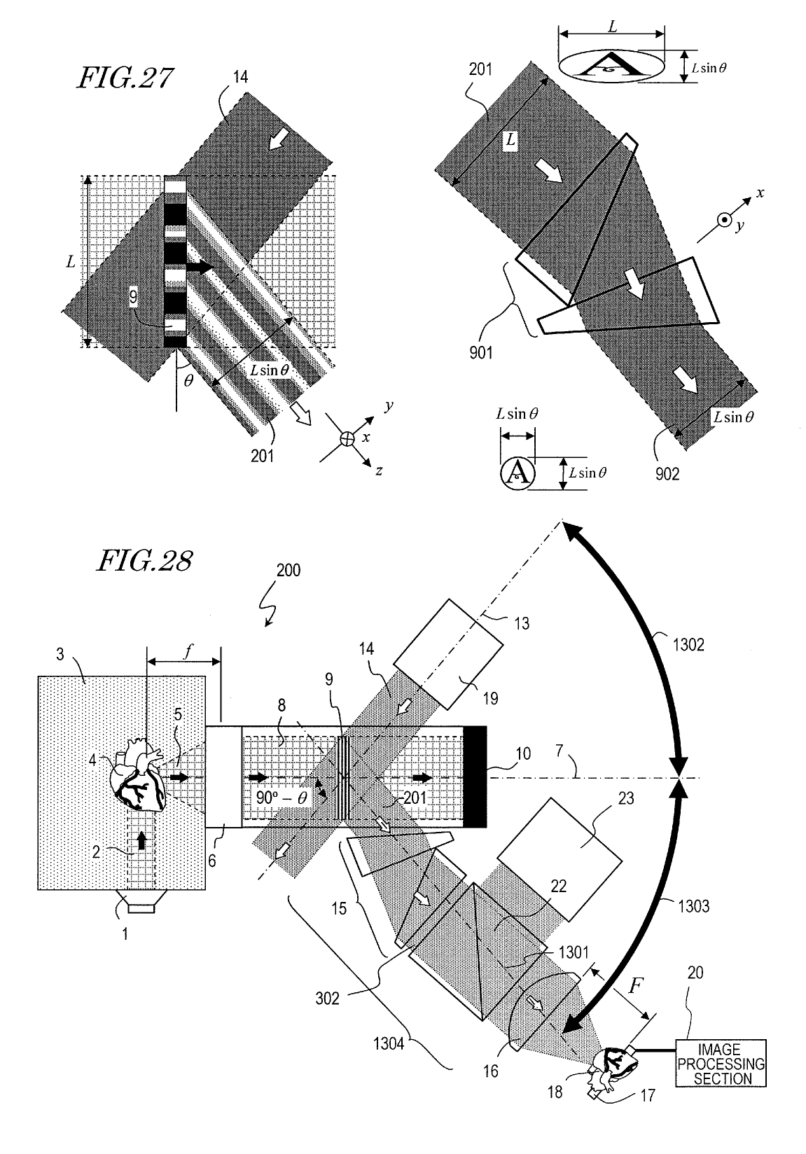

Hereinafter, a third embodiment of an acousto-optic vibrometer according to the present invention will be described. The acousto-optic vibrometer of this third embodiment is the same as the acousto-optic vibrometer 100 of the first embodiment except that the image distortion compensating section 15 has a different configuration. Thus, the following description of the third embodiment will be focused on only the configuration of the image distortion compensating section 15. FIG. 27 schematically illustrates a configuration for the image distortion compensating section 15 of this embodiment.

Supposing the angle of diffraction of a diffracted ray of light is θ (which is just as defined in the foregoing description), the image distortion compensating section 15 of this embodiment includes an optical reduction system 901 which multiplies the light beam width of the diffracted light 201 by sin θ in the x-axis direction in the coordinate system that has been set in FIG. 25. If the bundle of plane acoustic waves 9 has a circular cross section with a diameter L, the cross-sectional shape of the bundle of diffracted rays of light 201 becomes an elliptical one, of which the size as measured in the x-axis direction is L and the size as measured in the y-axis direction is L×sin θ. Since the diffracted light 201 is multiplied by sin θ in the x-axis direction by the optical reduction system 901, the cross-sectional shape of the bundle of diffracted rays of light 902 that has been subjected to the distortion compensation becomes a circular one, of which the diameter is L×sin θ. Although the image distortion compensating section 15 corrects the diffracted light 201 into a light beam with a diameter L in the first embodiment described above, the diffracted light 201 is corrected into a light beam with a diameter L×sin θ according to this embodiment.

As in the first embodiment described above, suppose the focal length of the acoustic lens system 6 is f, the focal length of the imaging lens system 16 is F, the wavelength of the plane acoustic wave 9 which is an ultrasonic wave is λₐ, the wavelength of the sensing light beam 14 which is a monochromatic light beam is λ₀, and the angle of diffraction is θ in this embodiment, too. In that case, the cross-sectional shape of the diffracted light beam 902 that has been subjected to the distortion compensation will be a circular one, and therefore, the real image 18 will be similar to the object 4. Also, according to the Fourier optics, their ratio of magnification will be (λₐ×f)/(λ₀×F)×sin θ. However, since Equation (1) needs to be satisfied, the ratio of magnification becomes 1/2×(f/F) when the diffracted light 201 is +first-order diffracted light.

As can be seen, by adopting this optical reduction system 901, the ratio of magnification no longer depends on the wavelengths of an ultrasonic wave and a monochromatic light beam. That is why if the ratio of the focal lengths of the acoustic lens system 6 and imaging lens system 16 is selected so that f/F=2 is satisfied, a real image 18 of the same size as the object 4 can be obtained and an image of the object 4 can be obtained with high resolution. Furthermore, if f is shortened, then F also becomes shorter, and therefore, the overall size of this acousto-optic vibrometer can be cut down, too. In addition, since the beam diameter of the distortion-compensated diffracted light 201 becomes narrower, the aperture size of the imaging lens system 16 also becomes smaller, and the overall size of the device can be reduced. In addition, the imaging lens system 16 no longer needs to have high plane precision.

In the first embodiment described above, the ratio of magnification of the real image 18 to the object 4 is (F×λ₀)/(f×λₐ). As already described for the specific example shown in FIG. 21, however, the wavelength λₐ of the ultrasonic wave is actually much longer than the wavelength λ₀ of the monochromatic light beam. That is why to obtain a large real image 18, an imaging lens system 16 with a very long focal length needs to be used. As a result, the size of the acousto-optic vibrometer 100 should be increased or an imaging lens system 16 with a special structure (such as the Cassegrain type reflective optical system described above) should be used. On the other hand, according to this embodiment, by using the optical reduction system 901 as the image distortion compensating section 15, a real image 18 can be obtained with high resolution, even though an imaging lens system 16 with a small aperture size and a short focal length is used. In addition, the overall size of the acousto-optic vibrometer can be cut down, too.

Even though the optical reduction system 901 is implemented as an anamorphic prism in the embodiment described above, any other optical reduction system with a similar function may also be used.

Also, in the embodiment described above, if the bundle of plane acoustic waves 9 has a circular cross-sectional shape with a diameter L, a distortion-compensated diffracted light beam 902 with a circular beam cross-sectional shape with a diameter L×sin θ is obtained. However, even if correction is made so that the distortion-compensated diffracted light beam 902 has a circular beam cross-sectional shape with a diameter C×L (where C<1), increase in the focal length of the imaging lens system 16 can also be minimized and the resolution of shooting can also be increased. For example, two image distortion compensating sections 15 may be provided and an optical reduction system and an optical magnification system may be used in the x- and y-axis directions, respectively, in the coordinate system shown in FIG. 27. Specifically, in that case, a beam contraction ratio in the x-axis direction and a beam expansion ratio in the y-axis direction may be selected so that the distortion-compensated diffracted light beam 902 has a circular beam cross-sectional shape with a diameter C×L (where C<1).

Optionally, an acousto-optic vibrometer including the optical reduction system 901 of this embodiment and the image distortion compensating section 15 of the second embodiment may also be used. In that case, the beam contraction ratio of the optical reduction system 901 is set so that the distortion-compensated diffracted light 902 has an elliptical beam cross-sectional shape with a size C×L (where C<1) in the x-axis direction and a size L×sin θ in the y-axis direction in the coordinate system defined in FIG. 27. In this manner, the resolution of the image shot can be substantially equalized everywhere on the focal plane of the imaging lens system 16.

### Embodiment 4

Hereinafter, a fourth embodiment of an acousto-optic vibrometer according to the present invention will be described. The acousto-optic vibrometer of this fourth embodiment further includes angle adjusting sections 1302 and 1303 unlike the acousto-optic vibrometer 102 of the first embodiment described above. Since the other components work in the same way as their counterparts of the acousto-optic vibrometer 100 of the first embodiment, the following description of the fourth embodiment will be focused on those angle adjusting sections 1302 and 1303.

As shown in FIG. 28, an optical system consisting of an image distortion compensating section 15, an imaging lens system 16 and an image receiving section 17 will be referred to herein as a “diffracted light imaging optical system 1304”. Also, its optical axis 1301 is in the plane including the acoustic axis 7 and the optical axis 13 and is a line which is mirror-symmetric to the optical axis 13 with respect to the acoustic axis 7 as the axis of symmetry.

The acousto-optic vibrometer 200 of this embodiment includes an angle adjusting section 1302 which adjusts the angle defined by the optical axis 13 of the sensing light source 19 with respect to the acoustic axis 7 and an angle adjusting section 1303 which adjusts the angle defined by the optical axis 1301 of the diffracted light imaging optical system 1305 with respect to the acoustic axis 7. These two angle adjusting sections 1302 and 1303 operate in conjunction with each other and adjust the angles so that the angle defined by the optical axis 13 with respect to the acoustic axis 7 is always equal to the angle defined by the optical axis 1301 with respect to the acoustic axis 7.

As already described for the first embodiment, the angle of diffraction 90 degrees−θ defined by the diffracted light 201 with respect to the acoustic axis 7 is determined based on the frequency of the sinusoidal wave as the acoustic wave 2 and the wavelength of the light emitted from the monochromatic light source 11. That is why even if the frequency of the acoustic wave 2 changes, the acousto-optic vibrometer 200 of this embodiment can also shoot the object 4 by getting the angle of diffraction adjusted by the angle adjusting sections 1302 and 1303.

The frequency of the acoustic wave 2 that the acousto-optic vibrometer 200 has can be set freely. That is why even if the frequency of the sinusoidal wave that forms the acoustic wave 2 is changed, the real image 18 can also be generated. Since the same object 4 can be observed with acoustic waves 2 having different frequencies, the resolution of shooting can be changed. According to this feature, a low-resolution image of the object 4 can be obtained with a low-frequency acoustic wave 2 first, and then a high-resolution image of the object 4 can be obtained with high definition using a high-frequency acoustic wave 2. As a result, the shooting session can get done in a shorter time and the size of the image data can be cut down.

### Embodiment 5

Hereinafter, a fifth embodiment of an acousto-optic vibrometer according to the present invention will be described. The acousto-optic vibrometer of this fifth embodiment includes two optical systems, each including the imaging lens system and the image receiving section, which is a major difference from the acousto-optic vibrometer 100 of the first embodiment. Thus, the following description of this fifth embodiment will be focused on such a difference from the first embodiment.

In the first embodiment described above, by capturing an image of the object 4 and measuring the velocity vector distribution v(x) in two different frames (see FIG. 20A), a high-definition image is obtained and the displacement velocity distribution is measured at a time. In addition, by shooting the object 4 and measuring the velocity vector distribution v(x) at the same time and by reducing the speckle through image processing (see FIG. 20B), an image in which the speckle pattern has been reduced significantly and the displacement velocity distribution can be obtained much more quickly than before. According to the former method, however, the displacement velocity distribution sometimes cannot be measured sufficiently quickly. On the other hand, according to the latter method, the speckle pattern may have been reduced insufficiently from the image.

Thus, according to this embodiment, in order to obtain a speckle-pattern-free high-definition image and a displacement velocity distribution quickly at the same time, two optical systems, each including the imaging lens system and the image receiving section, are used to shoot the object and measure the velocity vector distribution v(x) either simultaneously or using the same diffracted wave.

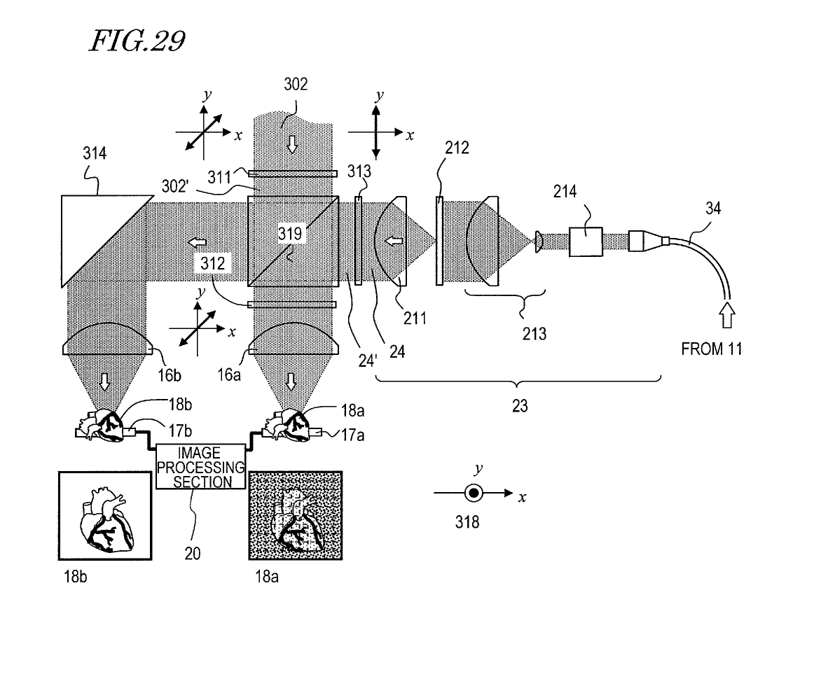

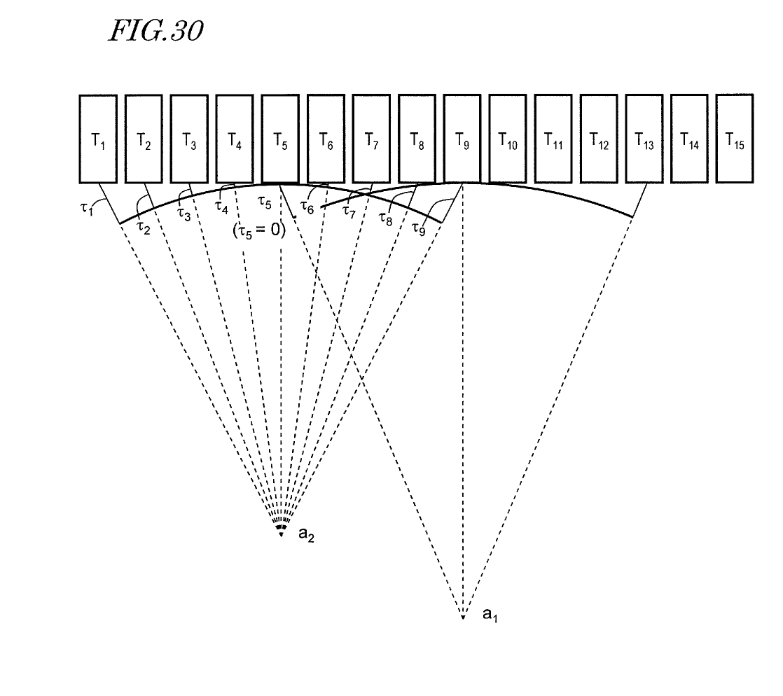

For that purpose, the acousto-optic vibrometer of this embodiment further includes polarizers 311, 312, 313 and polarization beam splitter 319 and also includes two imaging lens systems 16a, 16b and two image receiving sections 17a, 17b. As shown in FIG. 29, the reference light beam 24 emitted from the reference light source 23 is transformed into a linearly polarized light beam by the polarizer 313. When the coordinate system 318 is adopted as reference, the polarizer 313 is arranged so that the optic axis of the polarizer 313 becomes parallel to the y axis. As a result, the reference light beam 24′ of the linearly polarized light beam generated comes to have a polarization plane which is parallel to the y axis.

The polarization beam splitter 319 is configured to reflect only a linearly polarized light beam which is parallel to the y axis. All of the reference light beam 24′ is reflected from the polarization beam splitter 319 and incident on the image receiving section 17a via the imaging lens system 16a and does not reach the image receiving section 17b. The distortion-compensated diffracted light 302 is transformed by the polarizer 311 with an optic axis that is non-parallel to the y axis into linearly polarized diffracted light 302′. Since the polarization plane of the diffracted light 302′ is non-parallel to the y axis, the diffracted light 302′ is split by the polarization beam splitter 319 into a linearly polarized light beam, of which the polarization plane is parallel to the x-axis direction, and a linearly polarized light beam, of which the polarization plane is parallel to the y-axis direction. The linearly polarized light beam, of which the polarization plane is parallel to the x-axis direction, is transmitted through the polarization beam splitter 319 and travels toward the image receiving section 17a. On the other hand, the linearly polarized light beam, of which the polarization plane is parallel to the y-axis direction, is reflected from the polarization beam splitter 319, further reflected from the reflecting mirror 314, and then incident on the image receiving section 17b via the imaging lens system 16b.

Only the distortion-compensated diffracted light 302 is incident on the image receiving section 17b. That is why the real image 18b becomes a speckle-pattern-free optical image of the object 4. By getting the object 4 shot by the image receiving section 17b, an image of the object 4 of high image quality can be obtained.

On the other hand, the reference light beam 24′, of which the polarization plane is parallel to the y axis, is superposed on the distortion-compensated diffracted light 302′, of which the polarization plane is parallel to the x axis, and then incident on the image receiving section 17a. However, since the polarization planes of these two linearly polarized light beams intersect with each other at right angles, no interference occurs between them. That is why interference is produced by getting the two linearly polarized light beams transmitted through the polarizer 312, of which the optic axis is non-parallel to the y axis, and by matching the polarization planes of the linearly polarized light beams to each other. The light in which those two linearly polarized light beams are superposed one upon the other through the interference becomes a beat light beam. Based on the real image 18a detected at the image receiving section 17a, the velocity vector distribution v(x) is measured by the method that has already been described for the first embodiment. If the velocity vector distribution v(x) is measured as a three-dimensional vector quantity, the method that has been described with reference to FIG. 19 is used.

According to this embodiment, an acousto-optic vibrometer which can obtain a high-definition image of the object 4 and which can calculate a displacement velocity distribution quickly is realized.

An acousto-optic vibrometer according to the present disclosure can obtain an image of the object as an optical image using an acoustic wave, and therefore, can be used effectively as a probe for an ultrasonic diagnostic apparatus, for example. Also, even if the inside of an object that light cannot reach is going to be shot but if the object is made of a material that can propagate an ultrasonic wave, the distribution of moduli of elasticity inside the object can be observed as an optical image. That is why the acousto-optic vibrometer can also be used as a non-destructive vibrometer, for example. Furthermore, since the acousto-optic vibrometer can measure the displacement velocity of the object, the vibrometer can be used effectively as a non-contact vibrometer which measures a movement by a non-contact method or a vibration mode analyzer which measures an in-plane distribution of vibrations, for example.

while the present invention has been described with respect to preferred embodiments thereof, it will be apparent to those skilled in the art that the disclosed invention may be modified in numerous ways and may assume many embodiments other than those specifically described above. Accordingly, it is intended by the appended claims to cover all modifications of the invention that fall within the true spirit and scope of the invention.

What is claimed is:

1. An acousto-optic vibrometer comprising:
an acoustic wave source;
an acoustic lens system which places a scattered wave, created by irradiating an object with an acoustic wave that has been emitted from the acoustic wave source, into the scattered wave having a predetermined converged state;
an acousto-optic medium portion which is arranged so that the scattered wave that has been transmitted through the acoustic lens system is incident on the acousto-optic medium portion;
a sensing light source to emit a sensing light beam in which a plurality of monochromatic rays of light with mutually different traveling directions are superposed one upon the other and which is incident on the acousto-optic medium portion at an angle with respect to, and neither perpendicularly nor parallel to, the acoustic axis of the acoustic lens system;
a reference light source to emit a reference light beam which includes a plurality of monochromatic rays of light traveling into mutually different directions are superposed one upon the other and which is to be superposed on diffracted light that has been produced with the sensing light beam at the acousto-optic medium portion;
an imaging lens system which converges the diffracted light on which the reference light beam is superposed; and
an image receiving section which senses the light that has been converged by the imaging lens system to output an electrical signal.

2. The acousto-optic vibrometer of claim 1, wherein the sensing light beam and the reference light beam have mutually different frequencies.

3. The acousto-optic vibrometer of claim 1, wherein the reference light source includes at least one acousto-optic modulator.

4. The acousto-optic vibrometer of claim 3, wherein the reference light source includes a light scattering plate.

5. The acousto-optic vibrometer of claim 1, wherein the reference light source includes a fly-eye lens.

6. The acousto-optic vibrometer of claim 1, comprising two optical systems, each including the imaging lens system and the image receiving section.

7. The acousto-optic vibrometer of claim 6, wherein the reference light source includes a polarizer.

8. The acousto-optic vibrometer of claim 1, wherein the image receiving section is a two-dimensional image sensor with a plurality of pixels that are arranged two-dimensionally.

9. The acousto-optic vibrometer of claim 2, further comprising an image processing section which detects, based on the electrical signal, a variation with time in the quantity of light detected by each said pixel of the image receiving section.

10. The acousto-optic vibrometer of claim 1, wherein the reference light source includes a shutter which controls the time of emittance of the reference light beam.

11. The acousto-optic vibrometer of claim 1, wherein the acoustic wave source includes at least three acoustic wave sources.

12. The acousto-optic vibrometer of claim 1, further comprising an image distortion correcting section which corrects the distortion of the diffracted light and/or the distortion of the object image represented by the electrical signal.

13. The acousto-optic vibrometer of claim 11, wherein the image distortion correcting section includes an optical member which increases the cross-sectional area of the diffracted light.

14. The acousto-optic vibrometer of claim 11, wherein the image distortion correcting section includes an optical member which decreases the cross-sectional area of the diffracted light.

15. The acousto-optic vibrometer of claim 13, wherein the optical member includes an anamorphic prism.

16. The acousto-optic vibrometer of claim 13, wherein at least one of the imaging lens system and the optical member includes at least one cylindrical lens.

17. The acousto-optic vibrometer of claim 12, wherein the image distortion correcting section corrects the distortion of the object image, which is represented by the electrical signal, based on the electrical signal.

18. The acousto-optic vibrometer of claim 1, wherein the monochromatic rays of light have a spectrum width of less than 10 nm and are plane waves, of which the wavefront accuracy is ten times or less of the wavelength at the center frequency of the monochromatic rays of light.

19. The acousto-optic vibrometer of claim 1, wherein the sensing light source includes at least one fly-eye lens.

20. The acousto-optic vibrometer of claim 1, wherein the acoustic lens system includes at least one of a refracting acoustic lens and a reflecting acoustic lens.

21. The acousto-optic vibrometer of claim 20, wherein the acoustic lens system includes at least one acoustic element which is made of a material selected from the group consisting of a nanoporous silica, a fluorine-based inactive liquid, and polystyrene.

22. The acousto-optic vibrometer of claim 1, wherein the acoustic lens system includes at least one of a focal length adjusting mechanism and a focus position adjusting mechanism.

23. The acousto-optic vibrometer of claim 1, wherein the imaging lens system includes at least one of a focal length adjusting mechanism and a focus position adjusting mechanism.

24. The acousto-optic vibrometer of claim 1, wherein the acousto-optic medium portion includes at least one of a nanoporous silica, a fluorine-based inactive liquid, and water.

25. The acousto-optic vibrometer of claim 1, wherein the optical axis of the sensing light beam emitted from the sensing light source is adjustable with respect to the acoustic axis of the acoustic lens.
